(19) United States
(12) Patent Application Publication
(10) Pub. No.: US 2021/0257048 A1
(43) Pub. Date: Aug. 19, 2021

(54) METHODS AND SYSTEMS FOR CALLING MUTATIONS

(71) Applicant: Natera, Inc., San Carlos, CA (US)

(72) Inventors: Bernhard ZIMMERMANN, Manteca, CA (US); Raheleh SALARI, San Carlos, CA (US); Ryan SWENERTON, Millbrae, CA (US); Dina M. HAFEZ, Los Altos, CA (US)

(73) Assignee: Natera, Inc., San Carlos, CA (US)

(21) Appl. No.: 16/972,930

(22) PCT Filed: Jun. 12, 2019
(86) PCT No.: PCT/US2019/036718

§ 371 Date: Dec. 7, 2020

Related U.S. Application Data
(60) Provisional application No. 62/684,123, filed on Jun. 12, 2018.

(51) Int. Cl. G16B 20/20; C12Q 1/6886

(52) U.S. Cl. CPC G16B 20/20; C12Q 2600/156; C12Q 1/6886

(57) ABSTRACT

A method for calling a mutation includes determining, for each target base of a plurality of target bases, a respective value for a background error parameter based on training data. The method further includes determining a motif-specific error model including the background error parameter by performing processes that include: identifying a respective motif for each target base of the plurality of target bases, grouping the plurality of target bases into a plurality of groups, each group corresponding to a particular motif, and determining, for each group, a respective motif-specific parameter value for the background error parameter based on the determined values for the background error parameter for the target bases included in each group. The method further includes calling a mutation using the motif-specific error model and sequencing information for a biological sample.

### CROSS REFERENCE TO RELATED APPLICATIONS

This application claims priority to of U.S. Provisional Application No. 62/684,123, filed Jun. 12, 2018, which is hereby incorporated by reference in its entirety.

### BACKGROUND OF THE DISCLOSURE

Detecting mutations in genetic material can be used in tumor detection processes. For example, detection methods can be implemented to detect mutations such as single nucleotide variations (SNVs) or indels (insertions or deletions) at particular genetic positions that are correlated to the presence of tumors. In some implementations, recurrence monitoring is implemented by calling tumor specific mutations (e.g. making a determination that a mutation is present) in a subject's plasma that are contributed by circulating tumor DNA (ctDNA). Calling mutations can be based on a calling threshold that corresponds to a particular metric. For example, the calling threshold may be a threshold for a mutation fraction of the genetic targets, which is the percent of the genetic targets in a sample that differ from a reference allele. For example, if a reference or “normal” allele of a genetic target is a cytosine nucleotide (“C”), a mutation fraction would be a percent of the genetic targets that differ from C (e.g. that are an adenine (“A”), a thymine (“T”), or a guanine (“G”)). The mutation fraction may also refer to a fraction for a particular “channel” that refers to a target mutation being to a particular nucleotide (e.g., a target having a C reference allele may have three channels: C to A, C to G, and C to T, each having their own mutation fraction).

Mutation detection techniques typically involve performing a large number of test assays to generate target-specific statistics used for calling mutations (e.g. to account for errors, variance, or noise in the sequencing data). For example, a polymerase chain reaction (PCR) used to amplify genetic material extracted from a sample may introduce new mutations into the genetic material that were not present in a subject from whom the sample was extracted. This can be problematic for a mutation detection process that is meant to estimate a mutation fraction of an initial sample (prior to PCR). Thus, a large number of test assays including one or more PCRs can be performed to generate target-specific statistics and account for such errors. However, performing the large number of test assays for each desired target of a sequencing or testing process can be expensive and time consuming. It would be beneficial to avoid or omit the target-specific test assays. The present disclosure describes improved systems and methods that provide for, among other things, calling mutations without performing a large number of target-specific test assays.

### SUMMARY OF THE DISCLOSURE

At least some of the systems and methods described herein relate to determining a motif-specific error model that can be used in place of, or in addition to, target-specific test assays in a mutation-calling process. Motif refers to the sequence of the genome around or adjacent to the target location, and the motif error refers to the error for one specific base change of the motif. In some implementations, the error model can be determined using training data. Training data may be generated by sequencing of samples that have been processed using PCR, hybrid capture or other preparation procedures. Training data may include genetic segments that do not have, or are assumed to not have, mutations that would be expected if a tumor were present in the source of the sample. The training data may be generated from plasma samples. The training data may be generated from non-plasma samples. The training data may be generated from different workflows. For example, whole exome sequencing (WES) data, sequencing data following multiplex PCR (e.g., panel size of at least 100 genomic loci, at least 200 genomic loci, at least 500 genomic loci, at least 1,000 genomic loci, at least 2,000 genomic loci, at least 5,000 genomic loci, or at least 10,000 genomic loci), sequencing data following hybrid capture (e.g., panel size of at least 100 genomic loci, at least 200 genomic loci, at least 500 genomic loci, at least 1,000 genomic loci, at least 2,000 genomic loci, at least 5,000 genomic loci, or at least 10,000 genomic loci), as well as sequencing data of bespoke assays may be used to enhance the error model. In some embodiments, the workflow for training data and for sample analysis is generally the same. So for a PCR based assay, one can use the same workflow for training data. The training data do not have to come from analyzing the same target sequence and location as in the samples, but that the motif should be the same. The training data can be analyzed to generate results, reads, or counts of an error (e.g. a mutation, or a difference from a reference allele) detected after processing and sequencing. The training data can be used to characterize background error expected to be present in future assays performed on samples to call mutations. Background error may include any error that is present in an amplified sample (e.g. deviations from reference alleles) that is not due to mutations that were present in the initial control sample. For example, error induced during the sequencing, and/or error induced during the handling of biological samples may constitute background error. The background error may be characterized via one or more parameters, and the parameters may be included in the error model. For example, background error may be characterized, at least in part, as a background error parameter such as an amplification propagation error rate (rate at which errors are induced due to amplification).

In some implementations, the error determined from the training data can be specific to a group of bases at different positions having a same “motif.” A “motif” can be one or more bases adjacent to (either directly adjacent, or within a predetermined number of bases of) the target base. For example, a motif can include a base immediately prior to the target base in a genetic fragment being analyzed, and a base immediately following the target base in the genetic fragment being analyzed. Motifs may be symmetric or asymmetric. Other motif configurations may also be used, as described in more detail herein. The motif (e.g. the surrounding or adjacent bases) may influence background error, such as the error rate of the target base during sample processing, and thus similar error rates, or correlated error rates, may be expected for target bases having similar or identical motifs, even if the target bases are at different positions. Grouping the target bases that have a same motif and performing statistical analysis (e.g. the statistical analysis described herein) using the grouped target bases may provide for an improved estimate of the background error that can be applied in general fashion to targets having a same or similar motif. Thus, a motif-specific error model can be much more generalizable than a target-specific error model. By implementing the motif-specific error model, performing a large number of test assays for each target to generate target-specific statistics can be omitted, while still ensuring an accurate estimation of background error. Conventional systems and methods that do not implement the motif-specific approaches described herein are expensive and time consuming (e.g. due to the implementation of the test assays).

Accordingly, in one aspect, the present disclosure provides a method for calling a mutation. The method includes determining, for each target base of a plurality of target bases, a respective value for a background error parameter based on training data. The method further includes determining a motif-specific error model including the background error parameter by performing processes that include: identifying a respective motif for each target base of the plurality of target bases, grouping the plurality of target bases into a plurality of groups, each group corresponding to a particular motif, and determining, for each group, a respective motif-specific parameter value for the background error parameter based on the determined values for the background error parameter for the target bases included in each group. The method further includes calling a mutation using the motif-specific error model and sequencing information for a biological sample.

In another aspect, the present disclosure provides a system for calling a mutation. The system includes a processor, and computer memory storing machine-readable instructions that, when executed by the processor, cause the processor to determine, for each target base of a plurality of target bases, a respective value for a background error parameter based on training data, and determine a motif-specific error model including the background error parameter by performing processes that include identifying a respective motif for each target base of the plurality of target bases, grouping the plurality of target bases into a plurality of groups, each group corresponding to a particular motif, and determining, for each group, a respective motif-specific parameter value for the background error parameter based on the determined values for the background error parameter for the target bases included in each group. The machine-readable instructions, when executed by the processor, further cause the processor to call a mutation using the motif-specific error model and sequencing information for a biological sample.

In further aspect, the present disclosure provides a method for detecting a mutation associated with cancer, comprising: isolating cell-free DNA from a biological sample of a subject; amplifying from the isolated cell-free DNA a plurality of single-nucleotide variant (SNV) loci that comprise a plurality of target bases, wherein the SNV loci are known to be associated with cancer; sequencing the amplification products to obtain sequence reads of a plurality of motifs, wherein each motif comprises one of the plurality of target bases; determining a motif-specific background error parameter value; and identifying a mutation associated with cancer based on the motif-specific background error parameter value. In some embodiments, the biological sample is selected from blood, serum, plasma, and urine. In some embodiments, at least 10, or at least 20, or at least 50, or at least 100, or at least 200, or at least 500 SNV loci known to be associated with cancer are amplified from the isolated cell-free DNA. In some embodiments, the amplification products are sequenced with a depth of read of at least 200, or at least 500, or at least 1,000, or at least 2,000, or at least 5,000, or at least 10,000. In some embodiments, the plurality of single nucleotide variance loci are selected from SNV loci identified in the TCGA and COSMIC data sets for cancer.

In an additional aspect, the present disclosure provides a method for detecting a mutation associated with early relapse or metastasis of cancer, comprising: isolating cell-free DNA from a biological sample of a subject who has received treatment for a cancer; performing a multiplex amplification reaction to amplify from the isolated cell-free DNA a plurality of single-nucleotide variant (SNV) loci that comprise a plurality of target bases, wherein the SNV loci are patient-specific SNV loci associated with the cancer for which the subject has received treatment; sequencing the amplification products to obtain sequence reads of a plurality of motifs, wherein each motif comprises one of the plurality of target bases; determining a motif-specific background error parameter value; and identifying a mutation associated with early relapse or metastasis of cancer based on the motif-specific background error parameter value. In some embodiments, the biological sample is selected from blood, serum, plasma, and urine. In some embodiments, the multiplex amplification reaction amplifies at least 8, or at least 16, or at least 32, or at least 64, or at least 128 patient-specific SNV loci associated with the cancer for which the subject has received treatment. In some embodiments, the amplification products are sequenced with a depth of read of at least 200, or at least 500, or at least 1,000, or at least 2,000, or at least 5,000, or at least 10,000. In some embodiments, the method comprising collecting and analyzing a plurality of biological samples from the patient longitudinally.

The foregoing general description and following description of the drawings and detailed description are by way of example and explanatory and are intended to provide further explanation of the implementations as claimed. Other objects, advantages, and novel features will be readily apparent to those skilled in the art from the following brief description of the drawings and detailed description.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing.

FIG. 1 is a flow-chart illustrating a conventional approach to mutation calling and a motif-specific approach to mutation calling.

FIG. 2 illustrates one or more implementations of modelling a sample preparation process.

FIG. 3 illustrates a block diagram of one or more implementations of an error analysis system.

FIG. 4 illustrates one or more implementations of a method for calling a mutation using a motif-specific error model.

FIG. 5 illustrates one or more implementations of a method for determining a mutation fraction.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

Some of the description herein refers to calculating, determining, or estimating a variance of a parameter value, or using the variance to calculate, determine, or estimate another value. It should be understood that a standard deviation or other similar statistical measure may be used instead of, or in addition to, a variance, as appropriate.

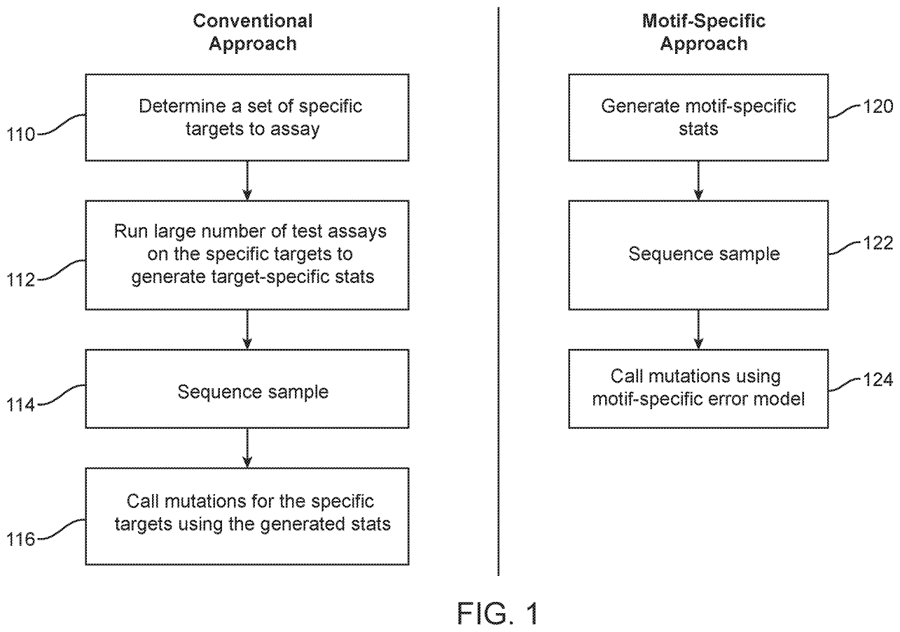

Referring now to FIG. 1, an illustration of a base-specific analysis and a motif-specific analysis of a sample are shown. The conventional approach includes at least four steps: determining a set of specific targets to assay (BLOCK 110), running a large number of test assays on the specific targets to generate target-specific statistics (BLOCK 112), sequencing a sample (BLOCK 114), and calling mutations for the specific targets using the generated statistics (BLOCK 116).

At BLOCK 110, a set of specific targets to be assayed is determine. Calling mutation using the conventional approach shown in FIG. 1 is limited to calling mutations for the specific targets determined at BLOCK 110. At BLOCK 112, dozens or hundreds of test assays may be performed for each target of interest (each target determined in BLOCK 110) to generate test data. For example, the test assays may include performing amplification process on genetic segments extracted from a test sample. The amplified segment may be exhaustively sequenced to generate background error statistics. For example, errors or mutations detected in the amplified result may be ascribed to errors induced by the amplification process, and an amplification propagation error rate may be estimated for the genetic sequences being assayed. A large number of test assays may be performed for each specific target to improve the estimate of the amplification propagation error rate.

At BLOCK 114, a genetic sample can be sequenced, and at BLOCK 116 mutations can be called using the determined amplification propagation error rate to account for at least some background error, and/or using other statistics generated at BLOCK 112. Mutations can only be called for the specific targets for which statistics were generated at BLOCK 112. Thus, to call mutations for a large number of targets of the sequenced sample, a very large number of test assays are performed, which can be expensive and time consuming.

The motif-specific approach improves on the conventional approach by providing for omission of the large number of target-specific test assays. Instead of generating target-specific statistics, an error model that provides for motif-specific statistics is used, which can be applied in a more general manner than can the target-specific approach (e.g. can be applied to any target having a same or similar motif as a motif used to generate test statistics). At BLOCK 120, using the methods and systems described herein, motif-specific statistics can be generated, which can constitute, or be used as part of, a motif-specific error model. Once a motif-specific error model has been established, the motif-specific approach can be implemented by sequencing a sample at BLOCK 122 and by calling mutations to targets having a specific motif using the motif-specific error model at BLOCK 124. The motif-specific error model has wide applicability. For example, a new sample can differ in at least some regards from a training sample used to generate the motif-specific error model, and it may be desirable to sequence targets for which no target-specific statistics exist (or for which existent statistics have an unacceptably or undesirably high degree of uncertainty). By using the motif-specific approach that leverages the tendency of background error to be motif-specific, the motif-specific error model can provide for accurate estimates of error associated with target bases in a sample that have a same motif as was analyzed and incorporated into the motif-specific error model, even though the target bases may be at different positions than the bases included in the training data used to generate the motif-specific error model. Thus, a large number of motif-specific test assays need not be performed for each sequencing and calling process for a sample to be sequenced. The motif-specific approach provides for accurate estimates of expected background error, which in turn can provide for highly accurate calling of mutations.

The present disclosure describes systems and methods that can be used to implement the motif-specific approach described above. The present disclosure describes statistical models, algorithms, and their implementation (e.g. for recurrence monitoring (RM)). RM can detect tumor specific mutations (targets) in a subject's plasma that are contributed by circulating tumor DNA (ctDNA). For that purpose targeted sequencing of a subject's plasma sample can be employed. Denoting the number of reads for a mutation at a certain position by E and the total number of reads at this position by X, and assuming that E comes from a Beta-Binomial distribution with parameters X and p(α, β)

E˜BB(X,p(α,β)) (1)

where p comes from Beta distribution with parameters α and β that are functions of replication efficiency and background error specific to sample preparation, these parameters can be estimated from a set of training samples with no mutations. In addition, these parameters are considered to be dependent on the fraction of ctDNA having the mutation, also called the real error as opposed to the background error generated during sample preparation and sequencing. Since the fraction of ctDNA present in the plasma sample may be unknown, α and β can be evaluated on a grid of values, and a mutation fraction that produces the highest probability for the data can be selected.

### Training or Sample Data Preparation

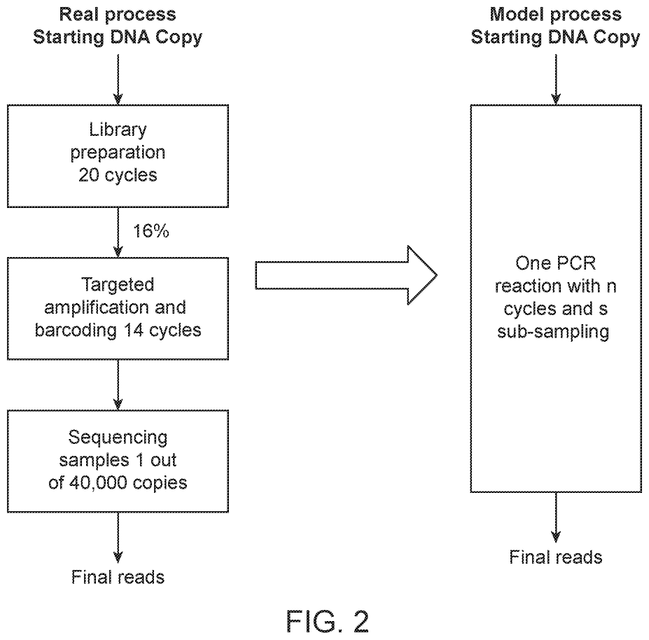

In some RM applications, samples are prepared in the lab in the course of two separate PCR reactions. After each reaction, only a portion of the product is passed to the next stage. This may be referred to as subsampling. To simplify computations, the present disclosure model the process by one PCR reaction with combined subsampling as illustrated in FIG. 2.

Some example implementations consider a total sub-sampling rate of 6×10⁻⁵ to model the process. The model assumes that a) the replication rate, or efficiency, p is constant from cycle to cycle; b) error rate pₑ is small compared to replication rate; c) an error occurs only once in the replication process, meaning that if a nucleotide base is substituted by another it will keep replicating unchanged for the rest of the process.

### Number of PCR Cycles

An RM variant calling algorithm estimates random SNV or indel error rate during the PCR reaction. The resulting frequency of PCR induced mutations depends on the number of PCR cycles that sample goes through. The number of cycles increases dynamically for samples with low initial DNA amounts as the saturation is reached later. Only the library preparation PCR reaction is affected by variable number of cycles. The starcoding reaction (targeted amplification and barcoding) is assumed to have the same number of cycles. Therefore, the total number of cycles is given by nₜₒₜₐₗ=n_{libprep}+n_{starcoding}. Based on the DNA input amount to library preparation step the algorithm estimates the total number of cycles to compute the expected PCR error more accurately. The number of cycles during library preparation is computed assuming the following starting_copies*(1+p)^{nlibprep}*libprep_loss=libprep_output_copies, where p is replication efficiency taken to be 0.9, libprep_loss is 0.75, libprep_output_copies=3*10⁶, and

$$starting\_copies = \frac{{x}_{i n p u t}}{3.3 * 1 {0}^{- 3}} ,$$

where xᵢₙₚᵤₜ is the DNA input amount in nanograms (ng). The n_{starcoding} is calibrated from the data to generate 10⁴ starting copies for samples with 33 ng input amount.

### Estimating a Mutation Fraction Distribution and Parameters

Estimating the above mentioned parameters α and β from the expectation and variance of the error rate can be implemented as follows. If μ is the expectation of the error rate after the PCR process and var is its variance as in

$$\begin{matrix}μ = 𝔼 ( \frac{E}{X} ) & ( 2 ) \\ var = 𝕍 ( \frac{E}{X} ) & ( 3 )\end{matrix}$$

then α and β of the corresponding Beta distribution are computed as

$$\begin{matrix}α = {μ}^{2} \frac{1 - μ}{var} - μ & ( 4 ) \\ β = α \frac{1}{μ} - 1 & ( 5 )\end{matrix}$$

The following expansion can be used to estimate μ and var

$$\begin{matrix}μ = 𝔼 ( \frac{E}{X} ) \approx \frac{𝔼 ( E )}{𝔼 ( X )} - \frac{Cov ( E , X )}{{( 𝔼 ( X ) )}^{2}} + \frac{𝔼 ( E ) V ( X )}{{( 𝔼 ( X ) )}^{3}} & ( 6 ) \\ var = 𝕍 ( \frac{E}{X} ) \approx \frac{𝕍 ( E )}{{( 𝔼 ( X ) )}^{2}} - \frac{2 𝔼 ( E ) Cov ( E , X )}{{( 𝔼 ( X ) )}^{3}} + \frac{{( 𝔼 ( E ) )}^{2} 𝕍 ( X )}{{( 𝔼 ( X ) )}^{4}} & ( 7 )\end{matrix}$$

Here, as defined above, X is the total number of reads and E is the number of reads for an error base, meaning the base that is different from the reference base. Since there are three possible changes from the reference (e.g. A can change to T, C, or G), there will be three expected error rates, one per each mutant base, or channel. The total error counts come from at least two sources—mutation in tumor DNA that is present before replication process and an erroneous substitution during the PCR process used in sample preparation. The former is referred to as the real error, and the latter as the background error.

E=E^{r}+E^{b} (8)

To determine a mutation fraction, or a probability distribution thereof, the replication efficiency and the probability of the background error per cycle is estimated from a set of training samples that are not expected to have any real mutations. Then, the starting count (or starting copy) is estimated based on the PCR efficiency. Using this estimate, the expectation and variance of total and error counts after the PCR process are computed, and can be plugged into Equations 6 and 7. Then, using Equations 4 and 5, the mutation fraction distribution parameters α and β can be determined.

### Modeling of the PCR Process and Useful Formulas

Assuming that at each PCR cycle n a) new DNA molecules are generated from the molecules present at the end of the previous cycle n−1 as governed by a binomial random process; b) molecules with a background error come from replication of errors from the previous cycle and new errors that occur at the current cycle randomly according the binomial random process with probability of error pₑ, having zero background errors present at the beginning of the PCR process; c) replication error occurs once per molecule and is not reversible; d) real errors are replicated with the same efficiency as normal molecules and their initial quantity is a fraction of the total molecules (e.g. if the starting copy is denoted by X₀ then there are f X₀ mutant molecules among them), then

Xₙ−Xₙ₋₁˜B(Xₙ₋₁,p)

Eₙ^{b}−Eₙ₋₁^{b}˜B((Xₙ₋₁−Eₙ₋₁^{b}),pₑ)+B(Eₙ₋₁^{b},p)

E₀^{r}=fX₀ (9)

Several values of f can be considered to find one that fits the data best.

### Expectation and Variance of Total Reads

From Equations 9, the expectation of the number of total reads conditioned on replication efficiency is given by

[symbol]

(Xₙ|p)= [symbol] (Xₙ₋₁|p)+p [symbol] (Xₙ₋₁|p)=(1+p)ⁿ [symbol] (X₀) (10)

The variance of this variable is given by

$$𝕍 ( {X}_{n} \vert p ) = p ( 1 - p ) 𝔼 ( {X}_{n - 1} \vert p ) + 𝕍 ( {X}_{n - 1} \vert p ) = ( 1 - p ) {( 1 + p )}^{n - 1} ( {( 1 + p )}^{n} - 1 ) 𝔼 ( {X}_{0} ) + {( 1 + p )}^{2 n} 𝕍 ( {X}_{0} ) \qquad ( 11 )$$

Here the last equality in each equation is produced by solving the recursive relation from the first part of the equation.

### Expectation and Variance for the Real Error Reads

Similarly to the total number of reads, for the real error the following equations apply:

[symbol]

(Eₙ^{r}|p)=f(1+p)ⁿ [symbol] (X₀)

[symbol]

(Eₙ^{r}|p)=f(1−p)(1+p)ⁿ⁻¹((1+p)ⁿ−1)) [symbol] (X₀)+f²(1+p)²ⁿ [symbol] (X₀) (12)

### Expectation and Variance for Background Error

For the sake of shortening the notations, in this section explicit reference to conditioning on p is omitted, but the statistics are conditional on p.

### Expectation of Background Error Reads

From Equations 9:

[symbol]

(Eₙ^{b}|Eₙ₋₁^{b}Xₙ₋₁)=(1+p)Eₙ₋₁^{b}+pₑ(Xₙ₋₁−Eₙ₋₁^{b})

which gives

$$𝔼 ( {E}_{n}^{b} ) = ( 1 + p - {p}_{e} ) 𝔼 ( {E}_{n - 1}^{b} ) + {p}_{e} 𝔼 ( {X}_{n - 1} ) = ( 1 + p - {p}_{e} ) 𝔼 ( {E}_{n - 1}^{b} ) + {{p}_{e} ( 1 + p )}^{n - 1} 𝔼 ( {X}_{0} )$$

where Equation 10 was used. Solving the recursive relation provides

$$𝔼 ( {E}_{n}^{b} ) = ( {( 1 + p )}^{n} - {( 1 + p - {p}_{e} )}^{n} ) 𝔼 ( {X}_{0} ) = {( 1 + p )}^{n} ( 1 - {( 1 - \frac{{p}_{e}}{1 + p} )}^{n} ) 𝔼 ( {X}_{0} )$$

For subsequent derivations, the approximation of this expression that comes from the equation above under the assumption that pₑ«p is used

[symbol]

(Eₙ^{b}≈npₑ(1+p)ⁿ⁻¹ [symbol] (X₀) (13)

### Variance of Background Error Reads

Some intermediate expressions that will be used in the following derivation are as follows:

[symbol]

(Eₙ^{b}|Eₙ₋₁^{b}Xₙ₋₁)=(1+p−pₑ)Eₙ₋₁^{b}+pₑXₙ₋₁ (14)

[symbol]

(Eₙ^{b}|Eₙ₋₁^{b}Xₙ₋₁)=(p(1−p)−pₑ(1−pₑ(1−pₑ))Eₙ₋₁^{b}+pₑ(1−pₑ)Xₙ₋₁ (15)

These follow directly from Equation 9. In deriving the last equation, the fact that Cov(B(Eₙ^{b},p),B(Xₙ−Eₙ^{b},pₑ)=0 was used.

With these, the variance term for the background error can be written as

$$𝕍 ( {E}_{n}^{b} ) = 𝔼 ( 𝕍 ( {E}_{n}^{b} \vert {E}_{n - 1}^{b} {X}_{n - 1} ) ) + 𝕍 ( 𝔼 ( {E}_{n}^{b} \vert {E}_{n - 1}^{b} {X}_{n - 1} ) ) == 𝔼 ( ( p ( 1 - p ) - {p}_{e} ( 1 - {p}_{e} ) ) {E}_{n - 1}^{b} + 𝔼 ( {p}_{e} ( 1 - {p}_{e} ) {X}_{n - 1} ) ++ 𝕍 ( ( 1 + p - {p}_{e} ) ) {E}_{n - 1}^{b} + {p}_{e} {X}_{n - 1} == ( p ( 1 - p ) - {p}_{e} ( 1 - {p}_{e} ) ) 𝔼 ( {E}_{n - 1}^{b} ) + {p}_{e} ( 1 - {p}_{e} ) 𝔼 ( {X}_{n - 1} ) ++ {p}_{e}^{2} 𝕍 ( {X}_{n - 1} ) + 2 {p}_{e} ( 1 + p - {p}_{e} ) Cov ( {E}_{n - 1}^{b} , {X}_{n - 1} ) + {( 1 + p - {p}_{e} )}^{2} 𝕍 ( {E}_{n - 1}^{b} ) \qquad ( 16 )$$

In the last equation, all terms except the last two have been computed. The very last term is used in a recursive relation that can provide the solution for variance. Thus the only term left to compute is the covariance.

The covariance term is computed separately since it is going to be useful by itself for the covariance of the total error with the total reads that enters Equations 6.

$$Cov ( {E}_{n}^{b} , {X}_{n} ) = 𝔼 ( Cov ( {E}_{n}^{b} , {X}_{n} \vert {E}_{n - 1}^{b} {X}_{n - 1} ) ++ Cov ( 𝔼 ( {E}_{b}^{n} \vert {E}_{n - 1}^{b} {X}_{n - 1} ) , 𝔼 ( {X}_{n} \vert {E}_{n - 1}^{b} {X}_{n - 1} ) ) == 𝔼 ( Cov ( {E}_{n - 1}^{b} + B ( {E}_{n - 1}^{b} , p ) + B ( {X}_{n - 1} - {E}_{n - 1}^{b} , {p}_{e} ) , {X}_{n - 1} + B ( {X}_{n - 1} , p ) \vert {E}_{n - 1}^{b} {X}_{n - 1} ) ) + Cov ( 𝔼 ( {E}_{n}^{b} \vert {E}_{n - 1}^{b} {X}_{n - 1} ) , 𝔼 ( {X}_{n} \vert {E}_{n - 1}^{b} {X}_{n - 1} ) ) = {T}_{1} + {T}_{2}$$

Here B( . . . ) stands for a random variable distributed according to binomial distribution with corresponding parameters, as defined in Equation 9. Two terms in the above equation are denoted by T₁ and T₂ and are computed separately below. For the next step in derivation, the expression

B(Xₙ₋₁,p)=B(Eₙ₋₁^{b},p)+B(Xₙ₋₁−Eₙ₋₁^{b},p)

is used, which holds if Xₙ₋₁ and Eₙ₋₁^{b} are constants as opposed to random variables. This is satisfied because these expressions enter conditional statistics. Using this, for the first term:

$${T}_{1} = 𝔼 ( Cov ( B ( {E}_{n - 1}^{b} , p ) , B ( {X}_{n - 1} , p ) \vert {E}_{n - 1}^{b} {X}_{n - 1} ) ++ Cov ( B ( {X}_{n - 1} - {E}_{n - 1}^{b} , {p}_{e} ) , B ( {x}_{n - 1} , P ) \vert {E}_{n - 1}^{b} {X}_{n - 1} == 𝔼 ( Cov ( B ( {E}_{n - 1}^{b} , p ) , B ( {E}_{n - 1}^{b} , p ) + B ( {X}_{n - 1} - {E}_{n - 1}^{b} , p ) \vert {E}_{n - 1}^{b} {X}_{n - 1} ) ++ Cov ( B ( {X}_{n - 1} - {E}_{n - 1}^{b} , {p}_{e} ) , B ( {E}_{n - 1}^{b} , p ) + B ( {X}_{n - 1} - {E}_{n - 1}^{b} , p ) \vert {E}_{n - 1}^{b} {x}_{n - 1} ) ) == 𝔼 ( Cov ( B ( {E}_{n - 1}^{b} , p ) , B ( {E}_{n - 1}^{b} , p ) \vert {E}_{n - 1}^{b} {X}_{n - 1} ) + Cov ( B ( {X}_{n - 1} - {E}_{n - 1}^{b} , {p}_{e} ) , B ( {X}_{n - 1} - {E}_{n - 1}^{b} , p ) \vert {E}_{n - 1}^{b} {X}_{n - 1} ) )$$

where the two crossed out terms amount to zero due to considerations for the physical process being modelled. The first crossed out term describes replication of error and normal molecules that, while conditioned on Xₙ₋₁ and Eₙ₋₁^{b}, is uncorrelated. The second crossed out term describes replication of error molecules and creation of new error molecules which are independent. Proceeding with evaluation of T₁:

$$\begin{matrix}{T}_{1} = 𝔼 ( 𝕍 ( B ( {E}_{n - 1}^{b} , p ) \vert {E}_{n - 1}^{b} {X}_{n - 1} ) + \\ + Cov ( B ( {X}_{n - 1} - {E}_{n - 1}^{b} , {p}_{e} ) , B ( {X}_{n - 1} - {E}_{n - 1}^{b} , p ) \vert {E}_{n - 1}^{b} {X}_{n - 1} ) ) \\ = p ( 1 - p ) 𝔼 ( {E}_{n - 1}^{b} ) + {p}_{e} ( 1 - p ) 𝔼 ( {X}_{n - 1} - {E}_{n - 1}^{b} )\end{matrix}$$

Here, the first term follows from the definition of variance for binomial distribution. The second term uses the following property: for two random binomial variables, Y and Z distributed as Y˜B(n, p) and Z˜B(Y, q) then

$$\begin{matrix}Cov ( Y , Z ) = 𝔼 ( YZ ) - 𝔼 ( Y ) 𝔼 ( Z ) = 𝔼 ( 𝔼 ( YZ \vert Y ) ) - np 𝔼 ( 𝔼 ( Z \vert Y ) = \\ = 𝔼 ( Y 𝔼 ( Z \vert Y ) ) - {n}^{2} {p}^{2} q = 𝔼 ( {qY}^{2} ) - {n}^{2} {p}^{2} q = \\ = q ( n p ( 1 - p ) + {n}^{2} {p}^{2} ) - {n}^{2} {p}^{2} q = qpn ( 1 - p )\end{matrix}$$

In the present case, Y represents the number of normal molecules replicating at cycle n−1 and Z—number of error molecules generated out of those molecules, and pₑ represents the probability of error given the probability of replication, so it is effectively p_{q} in the example above.

The second term, T₂ for the covariance expression is pretty straight forward.

$$\begin{matrix}{T}_{2} = Cov ( ( 1 + p - {p}_{e} ) {E}_{n - 1}^{b} + {p}_{e} {X}_{n - 1} , ( 1 + p ) {X}_{n - 1} ) = \\ = ( 1 + p ) ( 1 + p - {p}_{e} ) Cov ( {E}_{n - 1}^{b} , {X}_{n - 1} ) + {p}_{e} ( 1 + p ) 𝕍 ( {X}_{n - 1} )\end{matrix}$$

Putting together all the terms for covariance expression, a recursive relation is obtained:

Cov(Eₙ^{b},Xₙ)=(1+p)(1+p−pₑ)Cov(Eₙ₋₁^{b},Xₙ₋₁)+pₑ(1−p)(1+p)²ⁿ [symbol] (X₀)

Thus, a solution to the recursive relation in the following form would be useful:

aₙ=c₁aₙ₋₁+c₂d²⁽ⁿ⁻¹⁾+c₃(n−1)dⁿ⁻²

with
aₙ=Cov(Eₙ^{b},Xₙ)
c₁=(1+p)(1+p−pₑ)
c₂=Pₑ(1−p) [symbol] (X₀)+pₑ(1+p) [symbol] (X₀)
c₃+(p−pₑ)(1−p)pₑ [symbol] (X₀)
d=(1+p)
   After applying the recursive formula n times, the following pattern emerges:

$$\begin{matrix}{a}_{n} = {c}_{1}^{n} {a}_{0} + \\ + {c}_{2} ( {c}_{1}^{n - 1} + {c}_{1}^{n - 2} {d}^{2} + {{c}_{1}^{n - 3} ( {d}^{2} )}^{2} + … + {{c}_{1} ( {d}^{2} )}^{n - 2} + {( {d}^{2} )}^{n - 1} ) + \\ + {c}_{3} \frac{\partial }{\partial d} ( {c}_{1}^{n - 1} + {c}_{1}^{n - 2} d + … + {c}_{1} {d}^{n - 2} + {d}^{n - 1} ) = \\ = {c}_{2} \frac{{c}_{1}^{n} - {( {d}^{2} )}^{n}}{{c}_{1} - {d}^{2}} + {c}_{3} \frac{\partial }{\partial d} \frac{{c}_{1}^{n} - {d}^{n}}{{c}_{1} - d}\end{matrix}$$

where the formula for the sum of geometric progression Sₙ=Σₖ₌₀ⁿs^{n-k}t^{k}=sⁿΣₖ₌₀ⁿ(t/s)^{k}=(sⁿ⁺¹−tⁿ⁺¹)/(s−t) was used. Substituting all the coefficients and simplifying the expression provides the answer for covariance between the background error counts and the total number of reads as

$$\begin{matrix}Cov ( {E}_{n}^{b} , {X}_{n} ) = {n ( 1 + p )}^{2 n - 2} {p}_{e} ( 1 - p ) 𝔼 ( {X}_{0} ) + \\ + n {( 1 + p )}^{2 n - 2} ( 1 + p ) {p}_{e} 𝕍 ( {X}_{0} ) + \\ + {( 1 + p )}^{2 n - 2} \frac{1 - p}{p - {p}_{e}} 𝔼 ( {X}_{0} ) - \\ - {( 1 + p )}^{n - 1} {p}_{e} \frac{1 - p}{p - {p}_{e}} 𝔼 ( {X}_{0} )\end{matrix} \qquad ( 17 )$$

Substituting Equation 17 back into Equation 16 and grouping similar terms, the recursive relation for the variance is

[symbol]

(Eₙ^{b})=c₁ [symbol] (eₙ₋₁^{b})+c₂(1+p)ⁿ⁻¹+c₃(n−1)(1+p)ⁿ⁻²++c₄(1+p)²⁽ⁿ⁻¹⁾+c₅(n−1)(1+p)²ⁿ⁻⁴

with coefficients in this expression defined as

$$\begin{matrix}{c}_{1} = {( 1 + p )}^{2} - 2 ( 1 + p ) {p}_{e} + {p}_{e}^{2} \\ {c}_{2} = ( {p}_{e} - {p}_{e}^{2} - \frac{{p}_{e}^{2} ( 1 - p ( p + 2 )}{p ( 1 + p )} ) 𝔼 ( {X}_{0} ) \\ {c}_{3} = ( {p}_{e} p ( 1 - p ) - {p}_{e}^{2} ) 𝔼 ( {X}_{0} ) \\ {C}_{4} = {p}_{e}^{2} 𝕍 ( {X}_{0} ) + {p}_{e}^{2} \frac{( 1 - p ) ( p + 2 )}{p ( 1 + p )} 𝔼 ( {X}_{0} ) \\ {c}_{5} = 2 {p}_{e}^{2} ( ( 1 - {p}^{2} ) 𝔼 ( {X}_{0} ) + {( 1 + p )}^{2} 𝕍 ( {X}_{0} ) )\end{matrix} \qquad ( 18 )$$

where only terms up to pₑ² are kept. Going through a similar process as for Cov to solve this recursive relation, the solution for the variance of background error

$$𝕍 ( {E}_{n}^{b} \vert {pp}_{e} ) = {c}_{2} \frac{{c}_{1}^{n} - {x}^{n}}{{c}_{1} - x} + {c}_{3} \frac{{c}_{1}^{n} - {x}^{n} - n {x}^{n - 1} ( {c}_{1} - x )}{{( {c}_{1} - x )}^{2}} \qquad ( 19 )$$

is obtained, where the coefficients defined above and notations

x=1+p

y=(1+p)²

are used.

### Overview of Some Implementations

The derivations in the previous sections produce quantities conditioned on replication efficiency per cycle p and error rate per cycle pₑ. In order to evaluate absolute quantity Q, the following equations can be used

[symbol]

(Q)= [symbol] ( [symbol] (Q|p))=∫₀¹ [symbol] ((Q|p)f(p)dp

[symbol]

(Q)= [symbol] ( [symbol] (Q|p))+ [symbol] ( [symbol] (Q|p))

where f(p) stands for distribution of p that is to be estimated from the data. To remove conditioning on Pₑ the mean and variance of error rate is estimated and used to evaluate expressions as pₑ=mean(pe) and pₑ²=var(pₑ)+mean(pₑ)². It is also useful to compute [symbol] (X₀) and [symbol] (X₀) from data. Sequencing data including reads at targeted positions in a genome can be used. The present description distinguishes between a reference read Rᵣ, counts for the base specified in the reference genome, and error reads Rₑ, counts for the bases different from reference. The total reads, then, are defined as R=R^{r}+Σ_{nonref} R^{e} With these definitions, the following can be implemented.
Estimation of Efficiency and Error from the Training Data

Using a set of normal samples that are not expected to have any cancer related mutation, the efficiency can be estimated from relation R=(1+p)ⁿX₀ at each position. Assuming that starting copy or count X₀ is the same for each position, and assigning some arbitrary (relatively high) efficiency p* to positions with number of reads R* in high percentile (e.g. 99^{th} percentile),

$$\frac{1 + p}{1 + p *} = \frac{{( R / {X}_{0} )}^{1 / n}}{{( R * / {X}_{0} )}^{1 / n}} ⇒ p = {( \frac{R}{R *} )}^{\frac{1}{n}} ( 1 + {p}^{*} ) - 1 \qquad ( 20 )$$

Using this estimate for efficiency, the error rate per cycle at each position can be estimated from Equation 13 as

$${p}_{e} = \frac{{R}^{e}}{{n ( 1 + p )}^{n - 1} {X}_{0}} = \frac{{R}^{e} ( 1 + p )}{n R} \qquad ( 21 )$$

The mean and standard deviation of these quantities are found for each position by computing the statistics over multiple normal samples supplied in the data set. These values are later combined over bases sharing the same motifs, as described in more detail herein, and can be saved to be used for calling mutations in different samples.

### Estimation of Starting Copy for a Test Sample

Using the mean and standard deviation of efficiency for each position found previously from normal samples, the starting copy at each position for a test sample can be estimated as

$${X}_{0} = {\int }_{0}^{1} \frac{R}{{( 1 + p )}^{n}} f ( p ) dp \qquad ( 22 )$$

where f(p)=B(α, β) is the beta distribution with parameters α and β found from mean and standard deviation of efficiency. The mean and standard deviation of X₀ over positions belonging to the same sequenced genetic fragment can be computed and assigned to each position in the fragment.

### Adjusting Efficiency for a Test Sample

In some implementations, an update or correction of the efficiency values can be performed based on the found staring copy according to

$$p = \int ( {( \frac{R}{{x}_{0}} )}^{1 / n} - 1 ) g ( {x}_{0} ) d {x}_{0} \qquad ( 23 )$$

where g(x0)=N(μ, σ) is normal distribution with mean and standard deviation found for starting copy at particular position.

### Training Algorithms

In order to determine the mutation fraction distribution, appropriate training can be used to estimate the distribution parameters.

### Base Specific Training

For base specific training, the model parameters for each base can be estimated separately in the target panel. A basic assumption of this training process is that each base in the panel has a certain amplification rate and error rate. For this training method to work, control samples from normal subjects can be used. For example, 20-30 normal samples to estimate model parameters using base specific training can be used. The below algorithm outlines a basic flowchart of a base specific error model.

Algorithm 1 Base specific training algorithm

```
Training: D_{i,k} = (R_{i,k} , RefAlleleᵢ, A_{i,k}, C_{i,k}, G_{i,k}, T_{i,k}) where i ∈ {1, 2, . . . , B} denotes a base and k ∈
{1, 2, . . . , n} denotes a sample, RefAlleleᵢ is the reference/wildtype allele for base i, R_{i,k} is the
total depth of reads, A_{i,k}, C_{i,k}, G_{i,k}, T_{i,k} are the number of reads from alleles A, C, G, T
respectively.
Test: D_{i,k}^{Test} = (Rᵢ^{Test}, RefAlleleᵢ, Aᵢ^{Test}, Cᵢ^{Test}, Gᵢ^{Test}, Tᵢ^{Test}) for i = 1, 2, . . . , B. Mutation call
confidence scores for non-reference alleles in the test set for all bases 1, 2, . . . , B.
for i = 1, 2, . . . , B do
1. Estimate efficiency and error from training data as explained above for base i, using
  the data D_{i,k}.
2. Estimate starting copy for base i for test data at base i, using methods described
  above;
3. Adjust efficiency parameter at base i using methods described above.
4. For a grid of values of θ ∈ [0, τₘₐₓ] (where τₘₐₓ is ideally 1 but for practical
  purpose, it suffices to set τₘₐₓ ≈ 0.15) of candidate mutation fractions, plug in the
  estimated efficiency and error parameters in equation (6) and (7) to compute the
  likelihood L(θ) of test data using the beta-binomial model in (1).
5. Find Maximum Likelihood Estimate of θ, \hat{θ}_{MLE}: = argmax_{θ}L(θ)
$\text{6. Compute confidence score as} C = \frac{L ( {\hat{θ}}_{MLE} )}{L ( {\hat{θ}}_{MLE} ) + L ( 0 )}$
```

### Motif-Specific Training

Motif-specific training are useful in part because the sequence context around the base of interest contributes to the PCR error rate. Thus an error model can be generated from training data for each 3-base motif such that a base of interest is always the middle base. Other motifs can be used alternatively or additionally. For example, a motif may include one or more adjacent bases on only one side of the target base, or may include a symmetric (equal) or an asymmetric (not equal) number of bases on the two sides of the target base. Any number of adjacent bases may be defined as a motif. The motif specific error model estimates the middle base error parameters for each motif keeping the flanking bases same (e.g. estimates the error parameters for ATA→ACA, GTC→GAC, etc.). For example, in some implementations the algorithm estimates the error for

AAAATC → AAAACC
GATCA → GACCA
GTGGC → GCGGC
. . .

Dynamic flanking bases may also be implemented, and motifs may be variable based on the sequence context. In some embodiments, the motif comprises 0, 1, 2, 3, 4, or 5 adjacent bases before the target base. In some embodiments, the motif comprises 0, 1, 2, 3, 4, or 5 adjacent bases after the target base.

### Estimating Parameters for Motifs

Some implementations include performing the following steps:
1. From the training set, remove (bases, channel) data pairs for error rates more than or equal to α, where α=min{a predetermined number (e.g. 0.2), a predetermined percentile of the error rates in the training sample (e.g. the 99^{th} percentile)}.
2. Compute per cycle error rate per base per channel.
3. Compute mean and variance per motif using a grouped or pooled mean and variance formula. For example if μ₁, μ₂, . . . , μₙ are the means and σ₁², σ₂², . . . , σₙ² are the variances error rates of bases that share the same motif, then the pooled mean and variance may be calculated as

$${μ}_{pooled} = \frac{1}{n} {\sum }_{i = 1}^{n} {μ}_{i} \\ {σ}_{pooled}^{2} = \frac{1}{n} {\sum }_{i = 1}^{n} {σ}_{i}^{2}$$

4. If there are multiple training runs, then the pooling can be done stepwise, first pooling samples in individual runs and then pooling all runs. While pooling runs, the error rates can be weighted by number of occurrences of the motif in the run. In other implementations, the error rates are averaged without weighting.
5. Since the efficiency is not necessarily a function of motif, the efficiency parameter for each motif need not be averaged separately. Instead the mean and variances of the efficiency parameter is averaged over all samples to come up with one prior estimate for efficiency parameters. This prior estimate is no-longer position dependent. In other implementations, the efficiency parameter may be determined on a motif-specific basis, similarly to the determination of the motif-specific error rates.

Some implementations include fitting a regression model of the estimated efficiency values using the amplicon GC content, temperature, and so forth, as covariates and using this model to estimate the prior parameters instead of using a constant prior.

Algorithm 2 Motif specific training algorithm

Training Data: D_{i,k} = (R_{i,k} , RefAlleleᵢ, A_{i,k}, C_{i,k}, G_{i,k}, T_{i,k}) where i ∈ {1, 2, . . . , B_{Training}} denotes a
base and k ∈ {1, 2, . . . , n} denotes a sample, RefAlleleᵢ is the reference/wildtype allele for base I,
R_{i,k} is the total depth of reads, A_{i,k}, C_{i,k}, G_{i,k}, T_{i,k} are the number of reads from alleles A, C, G, T
respectively. M_{i,k} denotes the motif for the i-th base in sample k where M_{i,k} ∈ [symbol] : = {X₁X₂X₃}
such that Xj ∈ {A, C, G, T}∀j
Test Data: D_{i,k}^{Test} = (Rᵢ^{Test}, RefAlleleᵢ, Aᵢ^{Test}, Cᵢ^{Test}, Gᵢ^{Test}, Tᵢ^{Test}, Mᵢ^{Test}) for i =
1, 2, . . . , B_{TestData}.
Result: Mutation call confidence scores for non-reference alleles in the test set for all bases
1, 2, . . . , B.

| | |
|---|---|
| for Training do | >Training Block |

```
1: 1. Let α = min{a predetermined threshold, a predetermined percentile of observed hetrates in
    the training data.
  2. ∀i = 1, 2, · · · , B_{Training}; ∀k = 1, 2, · · · , n, compute per cycle efficiency p_{i,k} and error rate
    pe, i,k using the data D_{i,k}. If hetrate is ≥ α for some (base, channel) combination, then skip
    error estimation for that combination.
  3. Group the bases by motifs such that bases sharing the same motif are assigned to same
    group, forming M groups.
  4. ∀m ∈ [symbol] , compute mean and variance of error rates for m using the grouped data.
  5. Pool all bases together to compute the mean and variance of the efficiency parameter.
```

| | |
|---|---|
| for i = 1, 2, · · · , B_{Test} do | >Test Block |

```
2: 1. If the motif for base i is mᵢ, use universal efficiency parameters from last step and error
    parameters for motif mᵢ for subsequent steps.
  2. Estimate starting copy for base i for test data at base i.
  3. Adjust efficiency parameter at base i.
  4. For a grid of values of θ ∈ [0, τₘₐₓ] (where τₘₐₓ is ideally 1 but for practical purpose, it
    suffices to set τₘₐₓ ≈ 0.15) for candidate mutation fractions, plug in the estimated efficiency
    and error parameters in equation (6) and (7) to compute the likelihood L(θ) of test data
    using the beta-binomial model in (1).
  5. Find Maximum Likelihood Estimate of θ, θ, \hat{θ}_{MLE} : = argmax_{θ}L(θ).
  $\text{6. Compute confidence score as} C = \frac{L ( {\hat{θ}}_{MLE} )}{L ( {\hat{θ}}_{MLE} ) + L ( 0 )}$
```

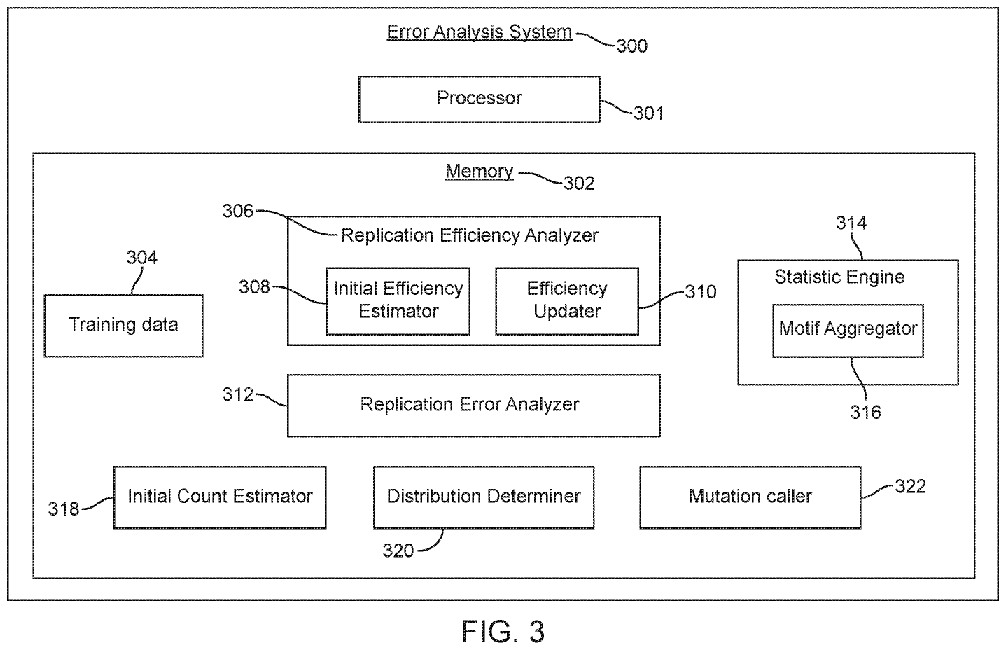

Referring now to FIG. 3, FIG. 3 is a block diagram showing an embodiment of an error analysis system 300. The error analysis system 300 can include one or more processors 301, and a memory 302. The one or more processors 301 may include one or more microprocessors, application-specific integrated circuits (ASIC), a field-programmable gate arrays (FPGA), etc., or combinations thereof. The memory 302 may include, but is not limited to, electronic, magnetic, or any other storage or transmission device capable of providing processor with program instructions. The memory may include magnetic disk, memory chip, read-only memory (ROM), random-access memory (RAM), Electrically Erasable Programmable Read-Only Memory (EEPROM), erasable programmable read only memory (EPROM), flash memory, or any other suitable memory from which processor can read instructions. The memory 302 may include components, subsystems, modules, scripts, applications, or one or more sets of processor-executable instructions for implementing error analysis processes, including any processes described herein. For example, the memory 302 may include training data 304, a replication efficiency analyzer 306, a replication error analyzer 312, a statistics engine 314, an initial count estimator 318, a distribution determiner 320, and a mutation caller 322.

The training data 304 can include, for example, data of the following type: (R_{i,k}, RefAlleleᵢ, A_{i,k}, C_{i,k}, G_{i,k}, T_{i,k}) where i∈{1, 2, . . . , B_{Training}} denotes a base and k∈{1, 2, . . . , n} denotes a sample, RefAlleleᵢ is the reference/wildtype allele for base I, R_{i,k} is the total depth of reads, A_{i,k}, C_{i,ki}, G_{i,k}, T_{i,k} are the number of reads from alleles A, C, G, T respectively. M_{i,k} denotes the motif for the i-th base in sample k where M_{i,k}∈ [symbol] :={X₁X₂X₃} such that Xⱼ∈{A, C, G, T}∀j. The training data may be derived from one or more one or more samples taken from one or more subjects. The training data may include only genetic material that does not include mutations of interest (e.g. mutations for which a mutation fraction is being determined).

The replication efficiency analyzer 306 may include components, subsystems, modules, scripts, applications, or one or more sets of processor-executable instructions for determining a replication efficiency of a PCR process, using the training data. The replication efficiency analyzer 306 may include an initial efficiency estimator 308 that determines an initial estimate of the replication efficiency. For example, the replication efficiency analyzer 306 may estimate the replication efficiency from the relation R=(1+p)ⁿX₀ at each position. The replication efficiency analyzer 306 may determine the initial replication efficiency estimate using Equation 20. The replication efficiency analyzer 306 may include an efficiency updater 310. The efficiency updater 310 may update or correct an initial efficiency estimate using an initial count determined by the initial count estimator 318 (described in more detail below). The efficiency updater 310 may update or correct the initial efficiency estimate using Equation 23.

The replication error analyzer 312 may include components, subsystems, modules, scripts, applications, or one or more sets of processor-executable instructions for determining a replication error rate. For example, the replication error analyzer 312 can determine an error rate per cycle at each position using equation 21. The determined error rate may correspond to background error, including error induced by the PCR process. The replication error analyzer 312 can determine the error rate per cycle at each position using the training data (e.g. based on the number of erroneous reads and the total number of reads made).

The statistics engine 314 may include components, subsystems, modules, scripts, applications, or one or more sets of processor-executable instructions for determining statistical values for the replication efficiencies determined by the replication efficiency analyzer 306, and for the replication error rates determined by the replication error analyzer 312. For example, the statistics engine 314 may determine a mean or estimated replication efficiency based on the replication efficiencies determined by the replication efficiency analyzer 306, and may determine a variance thereof. For example, the statistics engine 314 may determine the mean over all samples analyzed samples in a position-independent manner.

The statistics engine 314 may determine a mean or estimated replication error rate, and variance thereof, based on the replication error rates determined by the replication error analyzer 312. The mean or estimated replication error rate may be motif-specific. For example, the statistics engine 314 may include a motif aggregator 316 that groups the target bases to be analyzed by motif (that is, into groups in which all target bases of the group have a same motif). In some implementations, the motif aggregator 316 references a data structure that specifies motif parameters (e.g. a first number of adjacent bases sequentially prior to the target base, and a second number of adjacent bases sequentially following the target base) that define the motifs. For example, if a plurality of mean replication error rates μ₁, μ₂, . . . , μₙ and a plurality of variances thereof σ₁², σ₂², . . . , σₙ² are determined by the statistics engine 314 based on data determined by the replication error analyzer 312, the motif-specific grouped mean and variance may be calculated as

$${μ}_{pooled} = \frac{1}{n} {\sum }_{i = 1}^{n} {μ}_{i} \\ {σ}_{pooled}^{2} = \frac{1}{n} {\sum }_{i = 1}^{n} {σ}_{i}^{2}$$

The grouping can be done stepwise, first grouping samples in individual runs and then grouping all runs. While grouping runs, the error rates can be weighted by number of occurrences of the motif in the run. In other implementations, the error rates are averaged without weighting.

The statistics engine 314 may implement a filtering policy to sanitize the data. For example, the statistics engine 314 may remove from the training set (bases, channel) data pairs for error rates more than or equal to α, where α=min{a predetermined number (e.g. 0.2), a predetermined percentile of the error rates in the training sample (e.g. the 99^{th} percentile)}.

The initial count estimator 318 may include components, subsystems, modules, scripts, applications, or one or more sets of processor-executable instructions for determining an initial count of a target base for one or more samples. For example, the initial count estimator 318 may use Equation 22 to determine a plurality of initial count estimates for each base being analyzed. The initial count estimator 318 (or, in some implementations, the statistics engine 314) may determine a plurality of estimates or mean values for the initial count, and variances thereof, over positions belonging to a same sequenced genetic fragment, and may assign those values to each position in the genetic fragment. Those values may be used by the initial efficiency updater 310 to update an initial efficiency estimate, as described herein.

The distribution determiner 320 may include components, subsystems, modules, scripts, applications, or one or more sets of processor-executable instructions for determining parameters for a distribution representing a mutation fraction of one or more analyzed samples. For example, the distribution determiner 320 may determine parameters for a Beta Binomial distribution of the mutation fraction. The distribution determiner 320 may, for a grid of values of θ∈[0, τₘₐₓ] (where τₘₐₓ is ideally 1 but for practical purpose, it suffices to set τₘₐₓ≈0.15) for candidate mutation fractions, plug in the estimated efficiency and error parameters in to equation (6) and (7) to compute the likelihood L(θ) of test data using the beta-binomial model in (1). The distribution determiner 320 may select a highest likelihood mutation fraction as the determined mutation fraction for the one or more analyzed samples.

The mutation caller 322 may include components, subsystems, modules, scripts, applications, or one or more sets of processor-executable instructions for determining parameters for calling mutations. The mutation caller 322 may call mutations based on one or more parameter values being equal to, or above, a predetermined threshold. For example, the parameter values can include a mutation fraction, an absolute number of detected errors or mutations, or a number of standard deviations by which those parameter values deviate from a reference or mean value. The mutation caller 322 may also determine a confidence corresponding to the called mutation (e.g. based at least in part on a difference between the parameter value and the threshold).

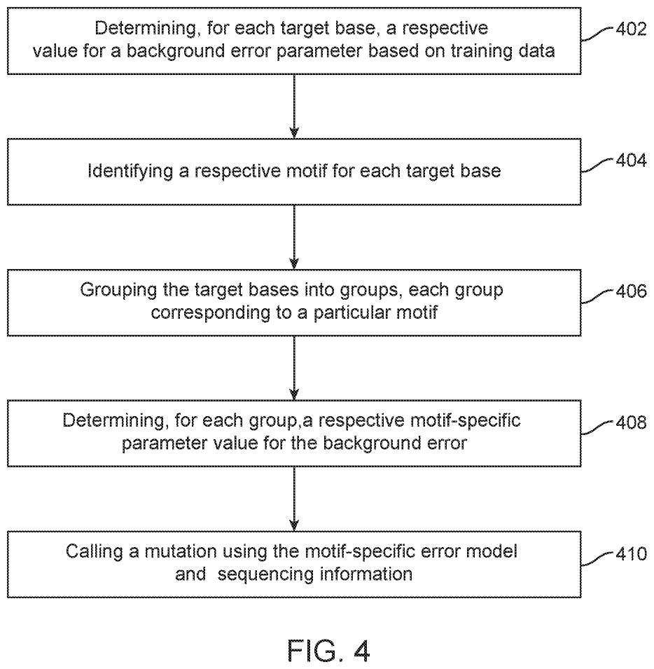

Referring now to FIG. 4, a method for calling a mutation using a motif-specific error model is shown. The method includes BLOCK 402 through BLOCK 410. In a brief overview, at BLOCK 402, the error analysis system 300 determines, for each target base of a plurality of target bases, a respective value for a background error parameter based on training data. At BLOCK 404, the error analysis system 300 identifies a respective motif for each target base. At BLOCK 406, the error analysis system 300 groups the target bases into groups, each group corresponding to a particular motif. At BLOCK 408, the error analysis system 300 determines, for each group, a respective motif-specific parameter value for the background error. At BLOCK 410, the error analysis system 300 calls a mutation using the motif-specific error model and sequencing information.

In more detail, at BLOCK 402, the error analysis system 300 determines, for each target base of a plurality of target bases, a respective value for a background error parameter based on training data. For example, the replication error analyzer 312 can determine an error rate per cycle for each target base of a plurality of target bases using equation 21. The determined error rate may correspond to background error, including error induced by the PCR process. The replication error analyzer 312 can determine the error rate per cycle at each position using the training data (e.g. based on the number of erroneous reads and the total number of reads made).

At BLOCK 404, the error analysis system 300 identifies a respective motif for each target base, and at BLOCK 406, the error analysis system 300 groups the target bases into groups, each group corresponding to a particular motif. For example, the motif aggregator 316 references a data structure that specifies motif parameters (e.g. a first number of adjacent bases sequentially prior to the target base, and a second number of adjacent bases sequentially following the target base) that define the motifs. For example, if a plurality of mean replication error rates μ₁, μ₂, . . . , μₙ and a plurality of variances thereof σ₁², σ₂², . . . , σₙ² are determined by the statistics engine 314 based on data determined by the replication error analyzer 312, the motif-specific grouped mean and variance may be calculated as

$${μ}_{pooled} = \frac{1}{n} {\sum }_{i = 1}^{n} {μ}_{i} \\ {σ}_{pooled}^{2} = \frac{1}{n} {\sum }_{i = 1}^{n} {σ}_{i}^{2}$$

The grouping can be done stepwise, first grouping samples in individual runs and then grouping all runs. While grouping runs, the error rates can be weighted by number of occurrences of the motif in the run. In other implementations, the error rates are averaged without weighting.

At BLOCK 408, the error analysis system 300 determines, for each group, a respective motif-specific parameter value for the background error. For example, the statistics engine 314 may determine a mean or estimated replication error rate, and variance thereof, for each group determined by the motif aggregator 316. Thus, the determined mean or estimated replication error rate may be motif-specific.

At BLOCK 410, the error analysis system 300 calls a mutation using the motif-specific error model and sequencing information. For example, the distribution determiner 320 may determine parameters for a Beta Binomial distribution of the mutation fraction. The distribution determiner 320 may, for a grid of values of θ∈[0, τₘₐₓ] (where τₘₐₓ is ideally 1 but for practical purpose, it suffices to set τₘₐₓ≈0.15) for candidate mutation fractions, plug in the estimated efficiency and error parameters in to equation (6) and (7) to compute the likelihood L(θ) of test data using the beta-binomial model in (1). The distribution determiner 320 may select a highest likelihood mutation fraction as the determined mutation fraction for the one or more analyzed samples. The mutation caller 322 may call mutations based on one or more parameter values being equal to, or above, a predetermined threshold. For example, the parameter values can include the mutation fraction determined by the distribution determiner 320. The mutation caller 322 may also determine a confidence corresponding to the called mutation (e.g. based at least in part on a difference between the parameter value and the threshold). Thus, a mutation can be accurately called using a motif-specific approach.

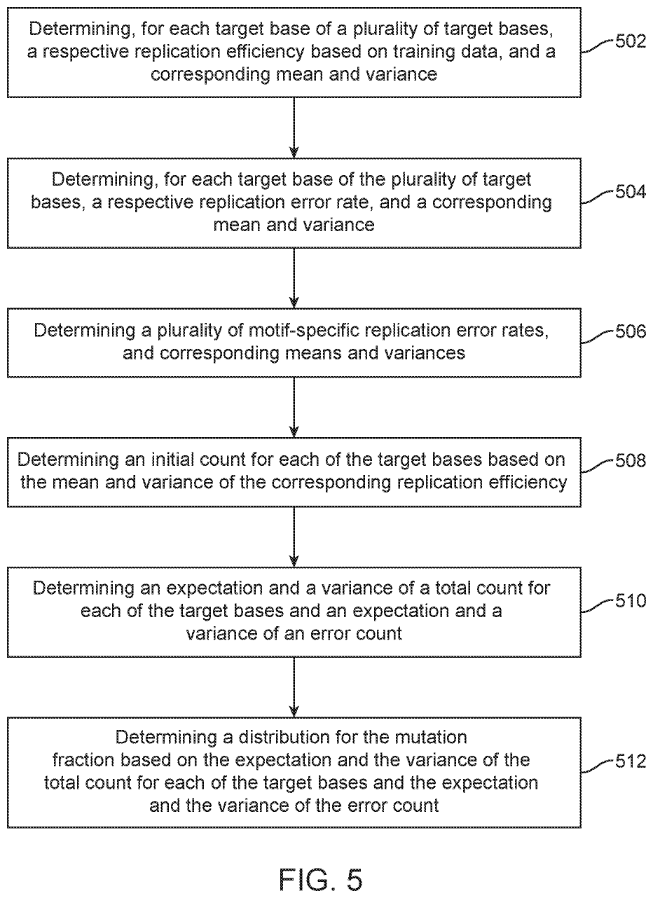

Referring now to FIG. 5, a method for determining a distribution for a mutation fraction is shown. The method includes BLOCK 502 through BLOCK 512. In a brief overview, at BLOCK 502, the error analysis system 300 determines, for each target base of a plurality of target bases, a respective replication efficiency based on training data, and a corresponding mean and variance. At BLOCK 504, the error analysis system 300 determines for each target base of the plurality of target bases, a respective replication error rate, and a corresponding mean and variance. At BLOCK 506, the error analysis system 300 determines a plurality of motif-specific replication error rates, and corresponding means and variances. At BLOCK 508, the error analysis system 300 determines an initial count for each of the target bases based on the mean and variance of the corresponding replication efficiency. At BLOCK 510, the error analysis system 300 determines an expectation and a variance of a total count for each of the target bases and an expectation and a variance of an error count. At BLOCK 512, the error analysis system 300 determines a distribution for the mutation fraction based on the expectation and the variance of the total count for each of the target bases and the expectation and the variance of the error count.

In more detail, at BLOCK 502, the replication efficiency analyzer 306 may determine an initial estimate of the replication efficiency. For example, the replication efficiency analyzer 306 may estimate the replication efficiency from the relation R=(1+p)ⁿX₀ at each position. The replication efficiency analyzer 306 may determine the initial replication efficiency estimate using Equation 20. The statistics engine 314 can determine corresponding mean values and variances.

At BLOCK 504, the replication error analyzer 312 may determine an error rate per cycle at each position using equation 21. The determined error rate may correspond to background error, including error induced by the PCR process. The replication error analyzer 312 can determine the error rate per cycle at each position using the training data (e.g. based on the number of erroneous reads and the total number of reads made). The statistics engine 314 can determine corresponding mean values and variances.

At BLOCK 506, the motif aggregator 316 may group the target bases to be analyzed by motif (that is, into groups in which all target bases of the group have a same motif). In some implementations, the motif aggregator 316 references a data structure that specifies motif parameters (e.g. a first number of adjacent bases sequentially prior to the target base, and a second number of adjacent bases sequentially following the target base) that define the motifs. The grouping can be done stepwise, first grouping samples in individual runs and then grouping all runs. While grouping runs, the error rates can be weighted by number of occurrences of the motif in the run. In other implementations, the error rates are averaged without weighting. The statistics engine 314 may determine motif-specific mean or estimated replication error rates, and variances thereof, based on the determined groups.

At BLOCK 508, the initial count estimator 318 may use Equation 22 to determine a plurality of initial count estimates for each base being analyzed. The initial count estimator 318 (or, in some implementations, the statistics engine 314) may determine a plurality of estimates or mean values for the initial count, and variances thereof, over positions belonging to a same sequenced genetic fragment, and may assign those values to each position in the genetic fragment. Those values may be used by the initial efficiency updater 310 to update an initial efficiency estimate, as described herein.

At BLOCK 510, the error analysis system 300 determines an expectation and a variance of a total count for each of the target bases and an expectation and a variance of an error count, and at BLOCK 512, the error analysis system 300 determines a distribution for the mutation fraction based on the expectation and the variance of the total count for each of the target bases and the expectation and the variance of the error count. This can include, for a grid of values of θ∈[0, τₘₐₓ] (where τₘₐₓ is ideally 1 but for practical purpose, it suffices to set Tₘₐₓ≈0.15) for candidate mutation fractions, plugging in the estimated efficiency and error parameters in equation (6) and (7) to compute the likelihood L(θ) of test data using the beta-binomial model in (1). The process can further include finding a Maximum Likelihood Estimate of θ, θ, \hat{θ}_{MLE}:=argmax_{θ}L(θ), and computing confidence score as

$$C = \frac{L ( {\hat{θ}}_{MLE} )}{L ( {\hat{θ}}_{MLE} ) + L ( 0 )} .$$

The distribution determiner 320 may select a highest likelihood mutation fraction, and may select the corresponding mutation fraction distribution as a mutation fraction distribution corresponding to an analyzed sample. Thus, a mutation fraction and a distribution thereof may be determined using a motif-specific approach

The above-described embodiments can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. For example, the error analysis system 300 can be executed on a computer or specialty logic system that includes one or more processors.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, an intelligent network (IN), or the Internet. Such networks may be based on any suitable technology and may operate according to any suitable protocol and may include wireless networks, wired networks, or fiber optic networks.

A computer employed to implement at least a portion of the functionality described herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer-readable storage medium (or multiple computer-readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the present disclosure discussed above. The computer-readable medium or media can be transportable, such that the program or programs stored thereon can be loaded onto one or more different computers or other processors to implement various aspects of the present disclosure as discussed above.

The terms “application” or “script” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present disclosure.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product.

### Method for Detection Cancer-Associated Mutations

In further aspect, the present disclosure provides a method for detecting a mutation associated with cancer, comprising: isolating cell-free DNA from a biological sample of a subject; amplifying from the isolated cell-free DNA a plurality of single-nucleotide variant (SNV) loci that comprise a plurality of target bases, wherein the SNV loci are known to be associated with cancer; sequencing the amplification products to obtain sequence reads of a plurality of motifs, wherein each motif comprises one of the plurality of target bases; and determining a mutation fraction distribution for each of the plurality of target bases and identifying a mutation associated with cancer based on the mutation fraction distribution. In some embodiments, the biological sample is selected from blood, serum, plasma, and urine. In some embodiments, at least 10, or at least 20, or at least 50, or at least 100, or at least 200, or at least 500, or at least 1,000 SNV loci known to be associated with cancer are amplified from the isolated cell-free DNA. In some embodiments, the amplification products are sequenced with a depth of read of at least 200, or at least 500, or at least 1,000, or at least 2,000, or at least 5,000, or at least 10,000, or at least 20,000, or at least 50,000, or at least 100,000. In some embodiments, the plurality of single nucleotide variance loci are selected from SNV loci identified in the TCGA and COSMIC data sets for cancer.

In an additional aspect, the present disclosure provides a method for detecting a mutation associated with early relapse or metastasis of cancer, comprising: isolating cell-free DNA from a biological sample of a subject who has received treatment for a cancer; performing a multiplex amplification reaction to amplify from the isolated cell-free DNA a plurality of single-nucleotide variant (SNV) loci that comprise a plurality of target bases, wherein the SNV loci are patient-specific SNV loci associated with the cancer for which the subject has received treatment; sequencing the amplification products to obtain sequence reads of a plurality of motifs, wherein each motif comprises one of the plurality of target bases; and determining a mutation fraction distribution for each of the plurality of target bases and identifying a mutation associated with early relapse or metastasis of cancer based on the mutation fraction distribution. In some embodiments, the biological sample is selected from blood, serum, plasma, and urine. In some embodiments, the multiplex amplification reaction amplifies at least 4, or at least 8, or at least 16, or at least 32, or at least 64, or at least 128 patient-specific SNV loci associated with the cancer for which the subject has received treatment. In some embodiments, the amplification products are sequenced with a depth of read of at least 200, or at least 500, or at least 1,000, or at least 2,000, or at least 5,000, or at least 10,000, or at least 20,000, or at least 50,000, or at least 100,000. In some embodiments, the method comprising collecting and analyzing a plurality of biological samples from the patient longitudinally.

The terms “cancer” and “cancerous” refer to or describe the physiological condition in animals that is typically characterized by unregulated cell growth. A “tumor” comprises one or more cancerous cells. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm's tumor.

In certain examples, the methods includes identifying a confidence value for each allele determination at each of the set of single nucleotide variance loci, which can be based at least in part on a depth of read for the loci. The confidence limit can be set at least 75%, 80%, 85%, 90%, 95%, 96%, 96%, 98%, or 99%. The confidence limit can be set at different levels for different types of mutations

In any of the methods for detecting SNVs herein that include a ctDNA SNV amplification/sequencing workflow, improved amplification parameters for multiplex PCR can be employed. For example, wherein the amplification reaction is a PCR reaction and the annealing temperature is between 1, 2, 3, 4, 5, 6, 7, 8, 9, or 10° C. greater than the melting temperature on the low end of the range, and 2, 3, 4, 5, 6, 7, 8, 9, 10, 11, 12, 13, 14 or 15° on the high end the range for at least 10, 20, 25, 30, 40, 50, 06, 70, 75, 80, 90, 95 or 100% the primers of the set of primers.

In certain embodiments, wherein the amplification reaction is a PCR reaction the length of the annealing step in the PCR reaction is between 10, 15, 20, 30, 45, and 60 minutes on the low end of the range, and 15, 20, 30, 45, 60, 120, 180, or 240 minutes on the high end of the range. In certain embodiments, the primer concentration in the amplification, such as the PCR reaction is between 1 and 10 nM. Furthermore, in exemplary embodiments, the primers in the set of primers, are designed to minimize primer dimer formation.

Accordingly, in an example of any of the methods herein that include an amplification step, the amplification reaction is a PCR reaction, the annealing temperature is between 1 and 10° C. greater than the melting temperature of at least 90% of the primers of the set of primers, the length of the annealing step in the PCR reaction is between 15 and 60 minutes, the primer concentration in the amplification reaction is between 1 and 10 nM, and the primers in the set of primers, are designed to minimize primer dimer formation. In a further aspect of this example, the multiplex amplification reaction is performed under limiting primer conditions.

A sample analyzed in methods of the present invention, in certain illustrative embodiments, is a blood sample, or a fraction thereof. Methods provided herein, in certain embodiments, are specially adapted for amplifying DNA fragments, especially tumor DNA fragments that are found in circulating tumor DNA (ctDNA). Such fragments are typically about 160 nucleotides in length.

It is known in the art that cell-free nucleic acid (e.g. cfDNA), can be released into the circulation via various forms of cell death such as apoptosis, necrosis, autophagy and necroptosis. The cfDNA, is fragmented and the size distribution of the fragments varies from 150-350 bp to >10000 bp. (see Kalnina et al. World J Gastroenterol. 2015 Nov. 7; 21(41): 11636-11653). For example the size distributions of plasma DNA fragments in hepatocellular carcinoma (HCC) patients spanned a range of 100-220 bp in length with a peak in count frequency at about 166 bp and the highest tumor DNA concentration in fragments of 150-180 bp in length (see: Jiang et al. Proc Natl Acad Sci USA 112:E1317-E1325).

In an illustrative embodiment the circulating tumor DNA (ctDNA) is isolated from blood using EDTA-2Na tube after removal of cellular debris and platelets by centrifugation. The plasma samples can be stored at −80° C. until the DNA is extracted using, for example, QIAamp DNA Mini Kit (Qiagen, Hilden, Germany), (e.g. Hamakawa et al., Br J Cancer. 2015; 112:352-356). Hamakava et al. reported median concentration of extracted cell free DNA of all samples 43.1 ng per ml plasma (range 9.5-1338 ng ml/) and a mutant fraction range of 0.001-77.8%, with a median of 0.90%.

Methods of the present invention in certain embodiments, typically include a step of generating and amplifying a nucleic acid library from the sample (i.e. library preparation). The nucleic acids from the sample during the library preparation step can have ligation adapters, often referred to as library tags or ligation adaptor tags (LTs), appended, where the ligation adapters contain a universal priming sequence, followed by a universal amplification. In an embodiment, this may be done using a standard protocol designed to create sequencing libraries after fragmentation. In an embodiment, the DNA sample can be blunt ended, and then an A can be added at the 3′ end. A Y-adaptor with a T-overhang can be added and ligated. In some embodiments, other sticky ends can be used other than an A or T overhang. In some embodiments, other adaptors can be added, for example looped ligation adaptors. In some embodiments, the adaptors may have tag designed for PCR amplification.

A number of the embodiments provided herein, include detecting the SNVs in a ctDNA sample. Such methods in illustrative embodiments, include an amplification step and a sequencing step (Sometimes referred to herein as a “ctDNA SNV amplification/sequencing workflow). In an illustrative example, a ctDNA amplification/sequencing workflow can include generating a set of amplicons by performing a multiplex amplification reaction on nucleic acids isolated from a sample of blood or a fraction thereof from an individual, such as an individual suspected of having cancer wherein each amplicon of the set of amplicons spans at least one single nucleotide variant loci of a set of single nucleotide variant loci, such as an SNV loci known to be associated with cancer; and determining the sequence of at least a segment of at each amplicon of the set of amplicons, wherein the segment comprises a single nucleotide variant loci. In this way, this exemplary method determines the single nucleotide variants present in the sample.

Exemplary ctDNA SNV amplification/sequencing workflows in more detail can include forming an amplification reaction mixture by combining a polymerase, nucleotide triphosphates, nucleic acid fragments from a nucleic acid library generated from the sample, and a set of primers that each binds an effective distance from a single nucleotide variant loci, or a set of primer pairs that each span an effective region that includes a single nucleotide variant loci. The single nucleotide variant loci, in exemplary embodiments, is one known to be associated with cancer. Then, subjecting the amplification reaction mixture to amplification conditions to generate a set of amplicons comprising at least one single nucleotide variant loci of a set of single nucleotide variant loci, preferably known to be associated with cancer; and determining the sequence of at least a segment of each amplicon of the set of amplicons, wherein the segment comprises a single nucleotide variant loci.

The effective distance of binding of the primers can be within 1, 2, 3, 4, 5, 6, 7, 8, 9, 10, 11, 12, 13, 14, 15, 20, 25, 30, 35, 40, 45, 50, 75, 100, 125, or 150 base pairs of a SNV loci. The effective range that a pair of primers spans typically includes an SNV and is typically 160 base pairs or less, and can be 150, 140, 130, 125, 100, 75, 50 or 25 base pairs or less. In other embodiments, the effective range that a pair of primers spans is 20, 25, 30, 40, 50, 60, 70, 75, 100, 110, 120, 125, 130, 140, or 150 nucleotides from an SNV loci on the low end of the range, and 25, 30, 40, 50, 60, 70, 75, 100, 110, 120, 125, 130, 140, or 150, 160, 170, 175, or 200 on the high end of the range.

Primer tails can improve the detection of fragmented DNA from universally tagged libraries. If the library tag and the primer-tails contain a homologous sequence, hybridization can be improved (for example, melting temperature (Tm) is lowered) and primers can be extended if only a portion of the primer target sequence is in the sample DNA fragment. In some embodiments, 13 or more target specific base pairs may be used. In some embodiments, 10 to 12 target specific base pairs may be used. In some embodiments, 8 to 9 target specific base pairs may be used. In some embodiments, 6 to 7 target specific base pairs may be used.

In one embodiment, Libraries are generated from the samples above by ligating adaptors to the ends of DNA fragments in the samples, or to the ends of DNA fragments generated from DNA isolated from the samples. The fragments can then be amplified using PCR, for example, according to the following exemplary protocol: 95° C., 2 min; 15×[95° C., 20 sec, 55° C., 20 sec, 68° C., 20 sec], 68° C. 2 min, 4° C. hold.

Many kits and methods are known in the art for generation of libraries of nucleic acids that include universal primer binding sites for subsequent amplification, for example clonal amplification, and for subsequence sequencing. To help facilitate ligation of adapters library preparation and amplification can include end repair and adenylation (i.e. A-tailing). Kits especially adapted for preparing libraries from small nucleic acid fragments, especially circulating free DNA, can be useful for practicing methods provided herein. For example, the NEXTflex Cell Free kits available from Bioo Scientific ( ) or the Natera Library Prep Kit (available from Natera, Inc. San Carlos, Calif.). However, such kits would typically be modified to include adaptors that are customized for the amplification and sequencing steps of the methods provided herein. Adaptor ligation can be performed using commercially available kits such as the ligation kit found in the AGILENT SURESELECT kit (Agilent, Calif.).

Target regions of the nucleic acid library generated from DNA isolated from the sample, especially a circulating free DNA sample for the methods of the present invention, are then amplified. For this amplification, a series of primers or primer pairs, which can include between 5, 10, 15, 20, 25, 50, 100, 125, 150, 250, 500, 1000, 2500, 5000, 10,000, 20,000, 25,000, or 50,000 on the low end of the range and 15, 20, 25, 50, 100, 125, 150, 250, 500, 1000, 2500, 5000, 10,000, 20,000, 25,000, 50,000, 60,000, 75,000, or 100,000 primers on the upper end of the range, that each bind to one of a series of primer binding sites.

Primer designs can be generated with Primer3 (Untergrasser A, Cutcutache I, Koressaar T, Ye J, Faircloth B C, Remm M, Rozen S G (2012) “Primer3—new capabilities and interfaces.” Nucleic Acids Research 40(15):e115 and Koressaar T, Remm M (2007) “Enhancements and modifications of primer design program Primer3.” Bioinformatics 23(10):1289-91) source code available at primer3.sourceforge.net). Primer specificity can be evaluated by BLAST and added to existing primer design pipeline criteria:

Primer specificities can be determined using the BLASTn program from the ncbi-blast-2.2.29+ package. The task option “blastn-short” can be used to map the primers against hg19 human genome. Primer designs can be determined as “specific” if the primer has less than 100 hits to the genome and the top hit is the target complementary primer binding region of the genome and is at least two scores higher than other hits (score is defined by BLASTn program). This can be done in order to have a unique hit to the genome and to not have many other hits throughout the genome.

The final selected primers can be visualized in IGV (James T. Robinson, Helga Thorvaldsdóttir, Wendy Winckler, Mitchell Guttman, Eric S. Lander, Gad Getz, Jill P. Mesirov. Integrative Genomics Viewer. Nature Biotechnology 29, 24-26 (2011)) and UCSC browser (Kent W J, Sugnet C W, Furey T S, Roskin K M, Pringle T H, Zahler A M, Haussler D. The human genome browser at UCSC. Genome Res. 2002 June; 12(6):996-1006) using bed files and coverage maps for validation.

Methods described herein, in certain embodiments, include forming an amplification reaction mixture. The reaction mixture typically is formed by combining a polymerase, nucleotide triphosphates, nucleic acid fragments from a nucleic acid library generated from the sample, a set of forward and reverse primers specific for target regions that contain SNVs. The reaction mixtures provided herein, themselves forming in illustrative embodiments, a separate aspect of the invention.

An amplification reaction mixture useful for the present invention includes components known in the art for nucleic acid amplification, especially for PCR amplification. For example, the reaction mixture typically includes nucleotide triphosphates, a polymerase, and magnesium. Polymerases that are useful for the present invention can include any polymerase that can be used in an amplification reaction especially those that are useful in PCR reactions. In certain embodiments, hot start Taq polymerases are especially useful. Amplification reaction mixtures useful for practicing the methods provided herein, such as AmpliTaq Gold master mix (Life Technologies, Carlsbad, Calif.), are available commercially.

Amplification (e.g. temperature cycling) conditions for PCR are well known in the art. The methods provided herein can include any PCR cycling conditions that result in amplification of target nucleic acids such as target nucleic acids from a library. Non-limiting exemplary cycling conditions are provided in the Examples section herein.

There are many workflows that are possible when conducting PCR; some workflows typical to the methods disclosed herein are provided herein. The steps outlined herein are not meant to exclude other possible steps nor does it imply that any of the steps described herein are required for the method to work properly. A large number of parameter variations or other modifications are known in the literature, and may be made without affecting the essence of the invention.

In certain embodiments of the method provided herein, at least a portion and in illustrative examples the entire sequence of an amplicon, such as an outer primer target amplicon, is determined. Methods for determining the sequence of an amplicon are known in the art. Any of the sequencing methods known in the art, e.g. Sanger sequencing, can be used for such sequence determination. In illustrative embodiments high throughput next-generation sequencing techniques (also referred to herein as massively parallel sequencing techniques) such as, but not limited to, those employed in MYSEQ (ILLUMINA), HISEQ (ILLUMINA), ION TORRENT (LIFE TECHNOLOGIES), GENOME ANALYZER ILX (ILLUMINA), GS FLEX+ (ROCHE 454), can be used for sequencing the amplicons produced by the methods provided herein.

High throughput genetic sequencers are amenable to the use of barcoding (i.e., sample tagging with distinctive nucleic acid sequences) so as to identify specific samples from individuals thereby permitting the simultaneous analysis of multiple samples in a single run of the DNA sequencer. The number of times a given region of the genome in a library preparation (or other nucleic preparation of interest) is sequenced (number of reads) will be proportional to the number of copies of that sequence in the genome of interest (or expression level in the case of cDNA containing preparations). Biases in amplification efficiency can be taken into account in such quantitative determination.

Target Genes. Target genes of the present invention in exemplary embodiments, are cancer-related genes, and in many illustrative embodiments, cancer-related genes. A cancer-related gene refers to a gene associated with an altered risk for a cancer or an altered prognosis for a cancer. Exemplary cancer-related genes that promote cancer include oncogenes; genes that enhance cell proliferation, invasion, or metastasis; genes that inhibit apoptosis; and pro-angiogenesis genes. Cancer-related genes that inhibit cancer include, but are not limited to, tumor suppressor genes; genes that inhibit cell proliferation, invasion, or metastasis; genes that promote apoptosis; and anti-angiogenesis genes.

An embodiment of the mutation detection method begins with the selection of the region of the gene that becomes the target. The region with known mutations is used to develop primers for mPCR-NGS to amplify and detect the mutation.

Methods provided herein can be used to detect virtually any type of mutation, especially mutations known to be associated with cancer and most particularly the methods provided herein are directed to mutations, especially SNVs, associated with cancer. Exemplary SNVs can be in one or more of the following genes: EGFR, FGFR1, FGFR2, ALK, MET, ROS1, NTRK1, RET, HER2, DDR2, PDGFRA, KRAS, NF1, BRAF, PIK3CA, MEK1, NOTCH1, MLL2, EZH2, TET2, DNMT3A, SOX2, MYC, KEAP1, CDKN2A, NRG1, TP53, LKB1, and PTEN, which have been identified in various lung cancer samples as being mutated, having increased copy numbers, or being fused to other genes and combinations thereof (Non-small-cell lung cancers: a heterogeneous set of diseases. Chen et al. Nat. Rev. Cancer. 2014 Aug. 14(8):535-551). In another example, the list of genes are those listed above, where SNVs have been reported, such as in the cited Chen et al. reference.

Other exemplary polymorphisms or mutations are in one or more of the following genes: TP53, PTEN, PIK3CA, APC, EGFR, NRAS, NF2, FBXW7, ERBBs, ATAD5, KRAS, BRAF, VEGF, EGFR, HER2, ALK, p53, BRCA, BRCA1, BRCA2, SETD2, LRP1B, PBRM, SPTA1, DNMT3A, ARID1A, GRIN2A, TRRAP, STAG2, EPHA3/5/7, POLE, SYNE1, C20orf80, CSMD1, CTNNB1, ERBB2. FBXW7, KIT, MUC4, ATM, CDH1, DDX11, DDX12, DSPP, EPPK1, FAM186A, GNAS, HRNR, KRTAP4-11, MAP2K4, MLL3, NRAS, RB1, SMAD4, TTN, ABCC9, ACVR1B, ADAM29, ADAMTS19, AGAP10, AKT1, AMBN, AMPD2, ANKRD30A, ANKRD40, APOBR, AR, BIRC6, BMP2, BRAT1, BTNL8, C12orf4, C1QTNF7, C20orf186, CAPRIN2, CBWD1, CCDCl30, CCDCl93, CD5L, CDCl27, CDCl42BPA, CDH9, CDKN2A, CHD8, CHEK2, CHRNA9, CIZ1, CLSPN, CNTN6, COL14A1, CREBBP, CROCC, CTSF, CYP1A2, DCLK1, DHDDS, DHX32, DKK2, DLEC1, DNAH14, DNAH5, DNAH9, DNASE1L3, DUSP16, DYNC2H1, ECT2, EFHB, RRN3P2, TRIM49B, TUBB8P5, EPHA7, ERBB3, ERCC6, FAM21A, FAM21C, FCGBP, FGFR2, FLG2, FLT1, FOLR2, FRYL, FSCB, GAB1, GABRA4, GABRP, GH2, GOLGA6L1, GPHB5, GPR32, GPX5, GTF3C3, HECW1, HIST1H3B, HLA-A, HRAS, HS3ST1, HS6ST1, HSPD1, IDH1, JAK2, KDM5B, KIAA0528, KRT15, KRT38, KRTAP21-1, KRTAP4-5, KRTAP4-7, KRTAP5-4, KRTAP5-5, LAMA4, LATS1, LMF1, LPAR4, LPPR4, LRRFIP1, LUM, LYST, MAP2K1, MARCH1, MARCO, MB21D2, MEGF10, MMP16, MORC1, MRE11A, MTMR3, MUC12, MUC17, MUC2, MUC20, NBPF10, NBPF20, NEK1, NFE2L2, NLRP4, NOTCH2, NRK, NUP93, OBSCN, OR11H1, OR2B11, OR2M4, OR4Q3, OR5D13, OR8I2, OXSM, PIK3R1, PPP2R5C, PRAME, PRF1, PRG4, PRPF19, PTH2, PTPRC, PTPRJ, RAC1, RAD50, RBM12, RGPD3, RGS22, ROR1, RP11-671M22.1, RP13-996F3.4, RP1L1, RSBN1L, RYR3, SAMD3, SCN3A, SEC31A, SF1, SF3B1, SLC25A2, SLC44A1, SLC4A11, SMAD2, SPTA1, ST6GAL2, STK11, SZT2, TAF1L, TAX1BP1, TBP, TGFBI, TIF1, TMEM14B, TMEM74, TPTE, TRAPPC8, TRPS1, TXNDC6, USP32, UTP20, VASN, VPS72, WASH3P, WWTR1, XPO1, ZFHX4, ZMIZ1, ZNF167, ZNF436, ZNF492, ZNF598, ZRSR2, ABL1, AKT2, AKT3, ARAF, ARFRP1, ARID2, ASXL1, ATR, ATRX, AURKA, AURKB, AXL, BAP1, BARD1, BCL2, BCL2L2, BCL6, BCOR, BCORL1, BLM, BRIP1, BTK, CARD11, CBFB, CBL, CCND1, CCND2, CCND3, CCNE1, CD79A, CD79B, CDC73, CDK12, CDK4, CDK6, CDK8, CDKN1B, CDKN2B, CDKN2C, CEBPA, CHEK1, CIC, CRKL, CRLF2, CSF1R, CTCF, CTNNA1, DAXX, DDR2, DOT1L, EMSY (C11orf30), EP300, EPHA3, EPHA5, EPHB1, ERBB4, ERG, ESR1, EZH2, FAM123B (WTX), FAM46C, FANCA, FANCC, FANCD2, FANCE, FANCF, FANCG, FANCL, FGF10, FGF14, FGF19, FGF23, FGF3, FGF4, FGF6, FGFR1, FGFR2, FGFR3, FGFR4, FLT3, FLT4, FOXL2, GATA1, GATA2, GATA3, GID4 (C17orf39), GNA11, GNA13, GNAQ, GNAS, GPR124, GSK3B, HGF, IDH1, IDH2, IGF1R, IKBKE, IKZF1, IL7R, INHBA, IRF4, IRS2, JAK1, JAK3, JUN, KAT6A (MYST3), KDM5A, KDM5C, KDM6A, KDR, KEAP1, KLHL6, MAP2K2, MAP2K4, MAP3K1, MCL1, MDM2, MDM4, MED12, MEF2B, MEN1, MET, MITF, MLH1, MLL, MLL2, MPL, MSH2, MSH6, MTOR, MUTYH, MYC, MYCL1, MYCN, MYD88, NF1, NFKBIA, NKX2-1, NOTCH1, NPM1, NRAS, NTRK1, NTRK2, NTRK3, PAK3, PALB2, PAX5, PBRM1, PDGFRA, PDGFRB, PDK1, PIK3CG, PIK3R2, PPP2R1A, PRDM1, PRKAR1A, PRKDC, PTCH1, PTPN11, RAD51, RAF1, RARA, RET, RICTOR, RNF43, RPTOR, RUNX1, SMARCA4, SMARCB1, SMO, SOCS1, SOX10, SOX2, SPEN, SPOP, SRC, STAT4, SUFU, TET2, TGFBR2, TNFAIP3, TNFRSF14, TOP1, TP53, TSC1, TSC2, TSHR, VHL, WISP3, WT1, ZNF217, ZNF703, and combinations thereof (Su et al., J Mol Diagn 2011, 13:74-84; DOI:10.1016/j.jmoldx.2010.11.010; and Abaan et al., “The Exomes of the NCI-60 Panel: A Genomic Resource for Cancer Biology and Systems Pharmacology”, Cancer Research, Jul. 15, 2013, which are each hereby incorporated by reference in its entirety). Exemplary polymorphisms or mutations can be in one or more of the following microRNAs: miR-15a, miR-16-1, miR-23a, miR-23b, miR-24-1, miR-24-2, miR-27a, miR-27b, miR-29b-2, miR-29c, miR-146, miR-155, miR-221, miR-222, and miR-223 (Calin et al. “A microRNA signature associated with prognosis and progression in chronic lymphocytic leukemia.” N Engl J Med 353:1793-801, 2005, which is hereby incorporated by reference in its entirety).

Amplification (e.g. PCR) Reaction Mixtures

Methods of the present invention, in certain embodiments, include forming an amplification reaction mixture. The reaction mixture typically is formed by combining a polymerase, nucleotide triphosphates, nucleic acid fragments from a nucleic acid library generated from the sample, a series of forward target-specific outer primers and a first strand reverse outer universal primer. Another illustrative embodiment is a reaction mixture that includes forward target-specific inner primers instead of the forward target-specific outer primers and amplicons from a first PCR reaction using the outer primers, instead of nucleic acid fragments from the nucleic acid library. The reaction mixtures provided herein, themselves forming in illustrative embodiments, a separate aspect of the invention. In illustrative embodiments, the reaction mixtures are PCR reaction mixtures. PCR reaction mixtures typically include magnesium.

In some embodiments, the reaction mixture includes ethylenediaminetetraacetic acid (EDTA), magnesium, tetramethyl ammonium chloride (TMAC), or any combination thereof. In some embodiments, the concentration of TMAC is between 20 and 70 mM, inclusive. While not meant to be bound to any particular theory, it is believed that TMAC binds to DNA, stabilizes duplexes, increases primer specificity, and/or equalizes the melting temperatures of different primers. In some embodiments, TMAC increases the uniformity in the amount of amplified products for the different targets. In some embodiments, the concentration of magnesium (such as magnesium from magnesium chloride) is between 1 and 8 mM.

The large number of primers used for multiplex PCR of a large number of targets may chelate a lot of the magnesium (2 phosphates in the primers chelate 1 magnesium). For example, if enough primers are used such that the concentration of phosphate from the primers is ˜9 mM, then the primers may reduce the effective magnesium concentration by ˜4.5 mM. In some embodiments, EDTA is used to decrease the amount of magnesium available as a cofactor for the polymerase since high concentrations of magnesium can result in PCR errors, such as amplification of non-target loci. In some embodiments, the concentration of EDTA reduces the amount of available magnesium to between 1 and 5 mM (such as between 3 and 5 mM).

In some embodiments, the pH is between 7.5 and 8.5, such as between 7.5 and 8, 8 and 8.3, or 8.3 and 8.5, inclusive. In some embodiments, Tris is used at, for example, a concentration of between 10 and 100 mM, such as between 10 and 25 mM, 25 and 50 mM, 50 and 75 mM, or 25 and 75 mM, inclusive. In some embodiments, any of these concentrations of Tris are used at a pH between 7.5 and 8.5. In some embodiments, a combination of KCl and (NH₄)₂SO₄ is used, such as between 50 and 150 mM KCl and between 10 and 90 mM (NH₄)₂SO₄, inclusive. In some embodiments, the concentration of KCl is between 0 and 30 mM, between 50 and 100 mM, or between 100 and 150 mM, inclusive. In some embodiments, the concentration of (NH₄)₂SO₄ is between 10 and 50 mM, 50 and 90 mM, 10 and 20 mM, 20 and 40 mM, 40 and 60 mM, or 60 and 80 mM (NH₄)₂SO₄, inclusive. In some embodiments, the ammonium [NH₄+] concentration is between 0 and 160 mM, such as between 0 to 50, 50 to 100, or 100 to 160 mM, inclusive. In some embodiments, the sum of the potassium and ammonium concentration ([K⁺]+[NH₄⁺]) is between 0 and 160 mM, such as between 0 to 25, 25 to 50, 50 to 150, 50 to 75, 75 to 100, 100 to 125, or 125 to 160 mM, inclusive. An exemplary buffer with [K⁺]+[NH₄⁺]=120 mM is 20 mM KCl and 50 mM (NH₄)₂SO₄. In some embodiments, the buffer includes 25 to 75 mM Tris, pH 7.2 to 8, 0 to 50 mM KCl, 10 to 80 mM ammonium sulfate, and 3 to 6 mM magnesium, inclusive. In some embodiments, the buffer includes 25 to 75 mM Tris pH 7 to 8.5, 3 to 6 mM MgCl₂, 10 to 50 mM KCl, and 20 to 80 mM (NH₄)₂SO₄, inclusive. In some embodiments, 100 to 200 Units/mL of polymerase are used. In some embodiments, 100 mM KCl, 50 mM (NH₄)₂SO₄, 3 mM MgCl₂, 7.5 nM of each primer in the library, 50 mM TMAC, and 7 ul DNA template in a 20 ul final volume at pH 8.1 is used.

In some embodiments, a crowding agent is used, such as polyethylene glycol (PEG, such as PEG 8,000) or glycerol. In some embodiments, the amount of PEG (such as PEG 8,000) is between 0.1 to 20%, such as between 0.5 to 15%, 1 to 10%, 2 to 8%, or 4 to 8%, inclusive. In some embodiments, the amount of glycerol is between 0.1 to 20%, such as between 0.5 to 15%, 1 to 10%, 2 to 8%, or 4 to 8%, inclusive. In some embodiments, a crowding agent allows either a low polymerase concentration and/or a shorter annealing time to be used. In some embodiments, a crowding agent improves the uniformity of the DOR and/or reduces dropouts (undetected alleles).

In some embodiments, a polymerase with proof-reading activity, a polymerase without (or with negligible) proof-reading activity, or a mixture of a polymerase with proof-reading activity and a polymerase without (or with negligible) proof-reading activity is used. In some embodiments, a hot start polymerase, a non-hot start polymerase, or a mixture of a hot start polymerase and a non-hot start polymerase is used. In some embodiments, a HotStarTaq DNA polymerase is used (see, for example, QIAGEN catalog No. 203203). In some embodiments, AmpliTaq Gold® DNA Polymerase is used. In some embodiments a PrimeSTAR GXL DNA polymerase, a high fidelity polymerase that provides efficient PCR amplification when there is excess template in the reaction mixture, and when amplifying long products, is used (Takara Clontech, Mountain View, Calif.). In some embodiments, KAPA Taq DNA Polymerase or KAPA Taq HotStart DNA Polymerase is used; they are based on the single-subunit, wild-type Taq DNA polymerase of the thermophilic bacterium Thermus aquaticus. KAPA Taq and KAPA Taq HotStart DNA Polymerase have 5′-3′ polymerase and 5′-3′ exonuclease activities, but no 3′ to 5′ exonuclease (proofreading) activity (see, for example, KAPA BIOSYSTEMS catalog No. BK1000). In some embodiments, Pfu DNA polymerase is used; it is a highly thermostable DNA polymerase from the hyperthermophilic archaeum Pyrococcus furiosus. The enzyme catalyzes the template-dependent polymerization of nucleotides into duplex DNA in the 5′→3′ direction. Pfu DNA Polymerase also exhibits 3′→5′ exonuclease (proofreading) activity that enables the polymerase to correct nucleotide incorporation errors. It has no 5′→3′ exonuclease activity (see, for example, Thermo Scientific catalog No. EP0501). In some embodiments Klentaq1 is used; it is a Klenow-fragment analog of Taq DNA polymerase, it has no exonuclease or endonuclease activity (see, for example, DNA POLYMERASE TECHNOLOGY, Inc, St. Louis, Mo., catalog No. 100). In some embodiments, the polymerase is a PHUSION DNA polymerase, such as PHUSION High Fidelity DNA polymerase (M0530S, New England BioLabs, Inc.) or PHUSION Hot Start Flex DNA polymerase (M0535S, New England BioLabs, Inc.). In some embodiments, the polymerase is a Q5® DNA Polymerase, such as Q5® High-Fidelity DNA Polymerase (M0491S, New England BioLabs, Inc.) or Q5® Hot Start High-Fidelity DNA Polymerase (M0493S, New England BioLabs, Inc.). In some embodiments, the polymerase is a T4 DNA polymerase (M0203S, New England BioLabs, Inc.).

In some embodiment, between 5 and 600 Units/mL (Units per 1 mL of reaction volume) of polymerase is used, such as between 5 to 100, 100 to 200, 200 to 300, 300 to 400, 400 to 500, or 500 to 600 Units/mL, inclusive.

PCR Methods. In some embodiments, hot-start PCR is used to reduce or prevent polymerization prior to PCR thermocycling. Exemplary hot-start PCR methods include initial inhibition of the DNA polymerase, or physical separation of reaction components reaction until the reaction mixture reaches the higher temperatures. In some embodiments, slow release of magnesium is used. DNA polymerase requires magnesium ions for activity, so the magnesium is chemically separated from the reaction by binding to a chemical compound, and is released into the solution only at high temperature. In some embodiments, non-covalent binding of an inhibitor is used. In this method a peptide, antibody, or aptamer are non-covalently bound to the enzyme at low temperature and inhibit its activity. After incubation at elevated temperature, the inhibitor is released and the reaction starts. In some embodiments, a cold-sensitive Taq polymerase is used, such as a modified DNA polymerase with almost no activity at low temperature. In some embodiments, chemical modification is used. In this method, a molecule is covalently bound to the side chain of an amino acid in the active site of the DNA polymerase. The molecule is released from the enzyme by incubation of the reaction mixture at elevated temperature. Once the molecule is released, the enzyme is activated.

In some embodiments, the amount to template nucleic acids (such as an RNA or DNA sample) is between 20 and 5,000 ng, such as between 20 to 200, 200 to 400, 400 to 600, 600 to 1,000; 1,000 to 1,500; or 2,000 to 3,000 ng, inclusive.

In some embodiments a QIAGEN Multiplex PCR Kit is used (QIAGEN catalog No. 206143). For 100×50 μl multiplex PCR reactions, the kit includes 2× QIAGEN Multiplex PCR Master Mix (providing a final concentration of 3 mM MgCl₂, 3×0.85 ml), 5×Q-Solution (1×2.0 ml), and RNase-Free Water (2×1.7 ml). The QIAGEN Multiplex PCR Master Mix (MM) contains a combination of KCl and (NH₄)₂SO₄ as well as the PCR additive, Factor MP, which increases the local concentration of primers at the template. Factor MP stabilizes specifically bound primers, allowing efficient primer extension by HotStarTaq DNA Polymerase. HotStarTaq DNA Polymerase is a modified form of Taq DNA polymerase and has no polymerase activity at ambient temperatures. In some embodiments, HotStarTaq DNA Polymerase is activated by a 15-minute incubation at 95° C. which can be incorporated into any existing thermal-cycler program.

In some embodiments, 1× QIAGEN MM final concentration (the recommended concentration), 7.5 nM of each primer in the library, 50 mM TMAC, and 7 ul DNA template in a 20 ul final volume is used. In some embodiments, the PCR thermocycling conditions include 95° C. for 10 minutes (hot start); 20 cycles of 96° C. for 30 seconds; 65° C. for 15 minutes; and 72° C. for 30 seconds; followed by 72° C. for 2 minutes (final extension); and then a 4° C. hold.

In some embodiments, 2× QIAGEN MM final concentration (twice the recommended concentration), 2 nM of each primer in the library, 70 mM TMAC, and 7 ul DNA template in a 20 ul total volume is used. In some embodiments, up to 4 mM EDTA is also included. In some embodiments, the PCR thermocycling conditions include 95° C. for 10 minutes (hot start); 25 cycles of 96° C. for 30 seconds; 65° C. for 20, 25, 30, 45, 60, 120, or 180 minutes; and optionally 72° C. for 30 seconds); followed by 72° C. for 2 minutes (final extension); and then a 4° C. hold.

Another exemplary set of conditions includes a semi-nested PCR approach. The first PCR reaction uses 20 ul a reaction volume with 2× QIAGEN MM final concentration, 1.875 nM of each primer in the library (outer forward and reverse primers), and DNA template. Thermocycling parameters include 95° C. for 10 minutes; 25 cycles of 96° C. for 30 seconds, 65° C. for 1 minute, 58° C. for 6 minutes, 60° C. for 8 minutes, 65° C. for 4 minutes, and 72° C. for 30 seconds; and then 72° C. for 2 minutes, and then a 4° C. hold. Next, 2 ul of the resulting product, diluted 1:200, is used as input in a second PCR reaction. This reaction uses a 10 ul reaction volume with 1× QIAGEN MM final concentration, 20 nM of each inner forward primer, and 1 uM of reverse primer tag. Thermocycling parameters include 95° C. for 10 minutes; 15 cycles of 95° C. for 30 seconds, 65° C. for 1 minute, 60° C. for 5 minutes, 65° C. for 5 minutes, and 72° C. for 30 seconds; and then 72° C. for 2 minutes, and then a 4° C. hold. The annealing temperature can optionally be higher than the melting temperatures of some or all of the primers, as discussed herein (see U.S. patent application Ser. No. 14/918,544, filed Oct. 20, 2015, which is herein incorporated by reference in its entirety).

The melting temperature (Tₘ) is the temperature at which one-half (50%) of a DNA duplex of an oligonucleotide (such as a primer) and its perfect complement dissociates and becomes single strand DNA. The annealing temperature (T_{A}) is the temperature one runs the PCR protocol at. For prior methods, it is usually 5° C. below the lowest Tₘ of the primers used, thus close to all possible duplexes are formed (such that essentially all the primer molecules bind the template nucleic acid). While this is highly efficient, at lower temperatures there are more unspecific reactions bound to occur. One consequence of having too low a T_{A} is that primers may anneal to sequences other than the true target, as internal single-base mismatches or partial annealing may be tolerated. In some embodiments of the present inventions, the T_{A} is higher than Tₘ, where at a given moment only a small fraction of the targets have a primer annealed (such as only ˜1-5%). If these get extended, they are removed from the equilibrium of annealing and dissociating primers and target (as extension increases Tₘ quickly to above 70° C.), and a new ˜1-5% of targets has primers. Thus, by giving the reaction a long time for annealing, one can get ˜100% of the targets copied per cycle.

In various embodiments, the annealing temperature is between 1, 2, 3, 4, 5, 6, 7, 8, 9, 10, 11, 12, 13° C. and 2, 3, 4, 5, 6, 7, 8, 9, 10, 11, 12, 13, or 15° C. on the high end of the range, greater than the melting temperature (such as the empirically measured or calculated Tₘ) of at least 25, 50, 60, 70, 75, 80, 90, 95, or 100% of the non-identical primers. In various embodiments, the annealing temperature is between 1 and 15° C. (such as between 1 to 10, 1 to 5, 1 to 3, 3 to 5, 5 to 10, 5 to 8, 8 to 10, 10 to 12, or 12 to 15° C., inclusive) greater than the melting temperature (such as the empirically measured or calculated Tₘ) of at least 25; 50; 75; 100; 300; 500; 750; 1,000; 2,000; 5,000; 7,500; 10,000; 15,000; 19,000; 20,000; 25,000; 27,000; 28,000; 30,000; 40,000; 50,000; 75,000; 100,000; or all of the non-identical primers. In various embodiments, the annealing temperature is between 1 and 15° C. (such as between 1 to 10, 1 to 5, 1 to 3, 3 to 5, 3 to 8, 5 to 10, 5 to 8, 8 to 10, 10 to 12, or 12 to 15° C., inclusive) greater than the melting temperature (such as the empirically measured or calculated Tₘ) of at least 25%, 50%, 60%, 70%, 75%, 80%, 90%, 95%, or all of the non-identical primers, and the length of the annealing step (per PCR cycle) is between 5 and 180 minutes, such as 15 and 120 minutes, 15 and 60 minutes, 15 and 45 minutes, or 20 and 60 minutes, inclusive.

Exemplary Multiplex PCR. In various embodiments, long annealing times (as discussed herein and exemplified in Example 12) and/or low primer concentrations are used. In fact, in certain embodiments, limiting primer concentrations and/or conditions are used. In various embodiments, the length of the annealing step is between 15, 20, 25, 30, 35, 40, 45, or 60 minutes on the low end of the range and 20, 25, 30, 35, 40, 45, 60, 120, or 180 minutes on the high end of the range. In various embodiments, the length of the annealing step (per PCR cycle) is between 30 and 180 minutes. For example, the annealing step can be between 30 and 60 minutes and the concentration of each primer can be less than 20, 15, 10, or 5 nM. In other embodiments the primer concentration is 1, 2, 3, 4, 5, 6, 7, 8, 9, 10, 15, 20, or 25 nM on the low end of the range, and 2, 3, 4, 5, 6, 7, 8, 9, 10, 15, 20, 25, and 50 on the high end of the range.

At high level of multiplexing, the solution may become viscous due to the large amount of primers in solution. If the solution is too viscous, one can reduce the primer concentration to an amount that is still sufficient for the primers to bind the template DNA. In various embodiments, between 1,000 and 100,000 different primers are used and the concentration of each primer is less than 20 nM, such as less than 10 nM or between 1 and 10 nM, inclusive.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations or implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description, or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. A method for calling a mutation, comprising:
determining, for each target base of a plurality of target bases, a respective value for a background error parameter based on training data;
determining a motif-specific error model including the background error parameter by performing processes that comprise:
identifying a respective motif for each target base of the plurality of target bases;
grouping the plurality of target bases into a plurality of groups, each group corresponding to a particular motif; and
determining, for each group, a respective motif-specific parameter value for the background error parameter based on the determined values for the background error parameter for the target bases included in each group; and
calling a mutation using the motif-specific error model and sequencing information for a biological sample.

2. The method of claim 1, wherein the background error parameter is a polymerase chain reaction (PCR) propagation error parameter.

3. The method of claim 1, wherein the respective motif for each target base of the plurality of target bases comprises a first number of bases prior to the target base, and a second number of bases following the target base.

4. The method of claim 3, wherein the first number and the second number are the equal.

5. The method of claim 4, wherein the first number is one and the second number is one.

6. The method of claim 3, further comprising determining the first number or the second number based on sequence context.

7. The method of claim 1, wherein the plurality of motif-specific background error parameter is specific to a change from a reference allele of the corresponding target base to a specific allele different from the target base.

8. The method of claim 1, wherein the training data comprises data for genetic segments having no mutations.

9. The method of claim 1, further comprising implementing a filtering policy that filters out one or more bases of the plurality of target bases having a replication error rate equal to, or exceeding, a predetermined threshold.

10. The method of claim 1, wherein calling a mutation based on the motif-specific error model comprises determining a respective mean and a respective variance for the motif-specific parameter value.

11. The method of claim 10, further comprising:
determining, using the training data, a mean replication efficiency replication and a variance of the replication efficiency; and
determining a mutation fraction based on the mean replication efficiency replication and the variance of the replication efficiency, and at least one of the respective mean and the respective variance for the motif-specific parameter value,
wherein calling the mutation is based on the determined mutation fraction.

12. The method of claim 11, further comprising determining an initial count for each of the target bases based on the mean and variance of the replication efficiency.

13. The method of claim 12, further comprising updating the determined replication efficiency based on the determined initial count.

14. The method of claim 13, further comprising determining a mean initial count and a variance of the initial count for a genetic segment of the biological sample based on a subset of the initial counts, and wherein the updating the determined replication efficiencies is based on the determined mean initial count and the determined variance of the initial count.

15. The method of claim 12, further comprising determining an expectation and a variance of a total count for each of the target bases and an expectation and a variance of an error count based on:
(i) the initial count for each of the target bases;
(ii) the mean and the variance of the replication efficiency; and
(iii) the mean and the variance of the motif-specific background error parameter value,
and wherein determining the mutation fraction is based on the expectation and the variance of the total count for each of the target bases and the expectation and the variance of the error count.

16. A method for detecting a mutation associated with cancer, comprising:
isolating cell-free DNA from the biological sample;
amplifying from the isolated cell-free DNA a plurality of single-nucleotide variant (SNV) loci that comprise a plurality of target bases, wherein the SNV loci are known to be associated with cancer;
sequencing the amplification products to obtain sequence reads of a plurality of motifs, wherein each motif comprises one of the plurality of target bases; and
determining a mutation fraction distribution for each of the plurality of target bases according to claim 1, and identifying a mutation associated with cancer based on the mutation fraction distribution.

17. The method according to claim 16, wherein the biological sample is selected from blood, serum, plasma, and urine.

18. The method according to claim 16, wherein at least 16 SNV loci known to be associated with cancer are amplified from the isolated cell-free DNA.

19. The method according to claim 16, wherein the amplification products are sequenced with a depth of read of at least 1,000.

20. The method according to claim 16, further comprising selecting the plurality of single nucleotide variance loci based on data corresponding to the biological sample.

21. A method for detecting a mutation associated with early relapse or metastasis of cancer, comprising:
isolating cell-free DNA from a biological sample of a subject who has received treatment for a cancer;
performing a multiplex amplification reaction to amplify from the isolated cell-free DNA a plurality of single-nucleotide variant (SNV) loci that comprise a plurality of target bases, wherein the SNV loci are patient-specific SNV loci associated with the cancer for which the subject has received treatment;
sequencing the amplification products to obtain sequence reads of a plurality of motifs, wherein each motif comprises one of the plurality of target bases; and
determining a mutation fraction distribution for each of the plurality of target bases according to claim 1, and identifying a mutation associated with early relapse or metastasis of cancer based on the mutation fraction distribution.

22. The method according to claim 21, wherein the biological sample is selected from blood, serum, plasma, and urine.

23. The method according to claim 21, wherein the multiplex amplification reaction amplifies at least 16 or at least 32 patient-specific SNV loci associated with the cancer for which the subject has received treatment.

24. The method according to claim 21, wherein the amplification products are sequenced with a depth of read of at least 1,000.

25. The method according to claim 21, wherein the method comprising collecting and analyzing a plurality of biological samples from the patient longitudinally.

26. A system for determining a mutation fraction distribution, comprising:
a processor; and
computer memory storing machine-readable instructions that, when executed by the processor, cause the processor to:
determine, for each target base of a plurality of target bases, a respective value for a background error parameter based on training data;
determine a motif-specific error model including the background error parameter by performing processes that comprise:
identifying a respective motif for each target base of the plurality of target bases;
grouping the plurality of target bases into a plurality of groups, each group corresponding to a particular motif; and
determining, for each group, a respective motif-specific parameter value for the background error parameter based on the determined values for the background error parameter for the target bases included in each group; and
call a mutation using the motif-specific error model and sequencing information for a biological sample.

27. The method of claim 26, wherein the background error parameter is a polymerase chain reaction (PCR) propagation error parameter.

28. The system of claim 26, wherein the respective motif for each target base of the plurality of target bases comprises a first number of bases prior to the target base, and a second number of bases following the target base.

29. The system of claim 28, wherein the first number and the second number are the equal.

30. The system of claim 29, wherein the first number is one and the second number is one.

31. The system of claim 28, wherein the machine-readable instructions, when executed by the processor, further cause the processor to determine the first number or the second number based on the sequence context.

32. The system of claim 27, wherein the plurality of motif-specific background error parameter is specific to a change from a reference allele of the corresponding target base to a specific allele different from the target base.

33. The system of claim 27, wherein the training data comprises data corresponding to genetic segments having no mutations.

34. The system of claim 27, wherein the machine-readable instructions, when executed by the processor, further cause the processor to implement a filtering policy that filters out one or more bases of the plurality of target bases having a replication error rate equal to, or exceeding, a predetermined threshold.

35. The system of claim 27, wherein the machine-readable instructions, when executed by the processor, further cause the processor to call the based on the motif-specific error model comprises determining a respective mean and a respective variance for the motif-specific parameter value.

36. The system of claim 35, wherein the machine-readable instructions, when executed by the processor, further cause the processor to:
determine, using the training data, a mean replication efficiency replication and a variance of the replication efficiency; and
determine a mutation fraction based on the mean replication efficiency replication and the variance of the replication efficiency, and at least one of the respective mean and the respective variance for the motif-specific parameter value,
wherein calling the mutation is based on the determined mutation fraction.

37. The system of claim 36, wherein the machine-readable instructions, when executed by the processor, further cause the processor to determine an initial count for each of the target bases based on the mean and variance of the replication efficiency.

38. The system of claim 37, wherein the machine-readable instructions, when executed by the processor, further cause the processor to update the determined replication efficiency based on the determined initial count.

39. The system of claim 38, wherein the machine-readable instructions, when executed by the processor, further cause the processor to determine a mean initial count and a variance of the initial count for a genetic segment of the biological sample based on a subset of the initial counts, and wherein the updating the determined replication efficiencies is based on the determined mean initial count and the determined variance of the initial count.

40. The system of claim 39, wherein the machine-readable instructions, when executed by the processor, further cause the processor to determine an expectation and a variance of a total count for each of the target bases and an expectation and a variance of an error count based on:
(i) the initial count for each of the target bases;
(ii) the mean and the variance of the replication efficiency; and
(iii) the mean and the variance of the motif-specific background error parameter value,
and wherein determining the mutation fraction is based on the expectation and the variance of the total count for each of the target bases and the expectation and the variance of the error count.
